(19) United States
(12) Patent Application Publication
(10) Pub. No.: US 2005/0118600 A1
(43) Pub. Date: Jun. 2, 2005

(54) Method for selecting drug sensitivity-determining factors and method for predicting drug sensitivity using the selected factors

(76) Inventors: Yuko Aoki, Kanagawa (JP); Kiyoshi Hasegawa, Kanagawa (JP); Nobuya Ishii, Kanagawa (JP); Kazushige Mori, Kanagawa (JP)

Correspondence Address: FISH & RICHARDSON PC, 225 FRANKLIN ST, BOSTON, MA, 02110, US

(21) Appl. No.: 10/507,389

(22) PCT Filed: Mar. 13, 2002
(86) PCT No.: PCT/JP02/02354

(51) Int. Cl. C12Q 1/68; G06F 19/00; G01N 33/48; G01N 33/50

(52) U.S. Cl. 435/6; 702/20

(57) ABSTRACT

Based on drug sensitivity data and extensive gene expression data, a model was constructed by multivariate analysis with the partial least squares method type 1. Further, the model was optimized using modeling power and genetic algorithm. Thereby, the degree of contribution of the respective genes to drug sensitivity was determined to select genes with a high degree of contribution. In addition, the levels of gene expression in specimens were analyzed, and then the drug sensitivity was predicted based on the model. The predicted values agreed well with those drug sensitivity values determined experimentally. The drug sensitivity-predicting method provided by the present invention enables assessment of the effectiveness of a drug prior to administration using small quantities of specimens associated with diseases such as cancer. Since this enables the selection of the most suitable drug for each patient, the present invention is very useful in improving a patient's quality of life (QOL).

### TECHNICAL FIELD

The present invention relates to a method for selecting drug sensitivity-determining factors using gene expression data and a method for predicting the drug sensitivity of unknown specimens using the determining factors selected. The present invention particularly relates to techniques for identifying genes that greatly contribute towards antitumor activity by revealing the correlation between antitumor effects and microarray data, and also techniques that predict antitumor effects of specimens with unknown sensitivity based on gene expression data.

### BACKGROUND ART

Although known anti-tumor drugs are not very effective in general, their side effects can be very serious and remarkably deteriorate a patient's quality of life (QOL). In order to improve the therapeutic effect and patients' QOL, it is necessary to predict the therapeutic effect an anti-cancer drug would have on a patient prior to the administration, and select an appropriate drug.

Since little is known about the sensitivity to drugs such as anti-tumor drugs, drugs are usually chosen through empirical decisions. Even though there is a drug-sensitivity test in which some cancer cells are obtained from a patient and tested for the sensitivity to various drugs in vitro, it is difficult to predict the sensitivity in vivo by this method, because of the difference between in vivo and in vitro environments, pharmacokinetic differences, and so forth. When preparing an antibody, since there is a correlation between the expression level of the antigen in cancer tissues and the effect, sensitive patients can be selected according to quantitative analysis based on the expression level in cancer tissues. On the other hand, in the case of a low molecular weight inhibitor, it is difficult to predict the sensitivity by analyzing a single molecule because cancer cells are heterogeneous and the target molecules are not only one.

In recent years, the emergence of the microarray technique has allowed extensive simultaneous gene expression analyses using small quantities of specimens. There are some attempts to predict the sensitivity according to this gene expression profile. However, when all the data obtained from the array are used, the predictability is very poor and thus, it is difficult to make an effective prediction.

Previously reported methods for selecting factors determining sensitivity include a method for estimating a group of genes, the expression levels of which differ between irradiation-sensitive and insensitive tumors, based on the clustering technique, which is one of the pattern recognition techniques (Hanna et al. (2001) Cancer Res. 61: 2376-2380). Also, a method comprising dividing specimens into two groups, namely a drug-sensitive group and an insensitive group, and selecting a group of genes, the expression levels of which are significantly different between the two groups using a test such as the U-test (Kihara et al. (2001) Cancer Res. 61: 6474-6479) has been reported. In this method, the sensitivity is then predicted by scoring the expression profile of genes selected based on the gene expression levels. These methods are based on the clustering and significant difference test, respectively, and both are only aimed at dividing the specimens into two groups, a drug-sensitive group and a drug-ineffective group. Thus, it is difficult to accurately predict the sensitivity by the methods. Further, these methods are not sufficient to quantitatively predict a value for sensitivity, namely the degree of effectiveness.

The number of genes on a microarray is overwhelmingly greater than that of specimens analyzed for gene expression, and the respective gene expression events are not independent of one another. Accordingly, it is difficult to successfully predict sensitivity with standard multivariate analyses such as simple regression analysis and multiple regression analysis used conventionally. Thus, the establishment of a method that precisely predicts drug sensitivity based on microarray data was required.

### DISCLOSURE OF THE INVENTION

The present invention provides a method for selecting drug sensitivity-determining genes using extensive gene expression data, high-density nucleic acid array to detect the expression of selected genes, and PCR probes and primers. The present invention further provides a method for predicting the drug sensitivity of unknown specimens using genes selected by the above method, and a computer device for predicting drug sensitivity. The method of the present invention allows the classification of unknown specimens and helps the planning of diagnostic and therapeutic methods based on drug sensitivity. Particularly, the present invention provides a method that specifies genes that greatly contribute towards the antitumor activity of a drug through revealing the correlation between the antitumor effect and microarray data, and further predicts the antitumor effect of the drug on specimens with unknown sensitivity based on the expression data of these genes.

Although it is essential in health care to develop techniques that quantitatively predict the antitumor effect of a particular drug prior to administration using gene expression data, such methods have not yet been developed. Using a novel multivariate analysis technique that can overcome the statistical constraints described above, the present inventors developed a model to accurately predict the sensitivity of specimens with unknown sensitivity by quantitatively determining a correlation between the antitumor effect and a gene expression profile. To achieve this object, the present inventors used the partial least squares method type 1 (PLS1), which is a novel multivariate analysis method that has been used in the fields of econometrics and chemometrics. This analysis method comprises deriving principal components from extensive gene expression data, such as microarray data, and drug sensitivity data, such as an antitumor effect, and subjecting the two principal components again to simple regression analysis. The use of principal components enabled the circumvention of the following statistical constraints: i) the respective gene expression events are not independent of one another; and ii) the number of genes is overwhelmingly greater than the number of specimens. PLS type 2 (PLS2) of the partial least squares method (PLS) enables one to identify important genes commonly affecting the sensitivity to drugs based on, for example, the relationship between the cells and expression of multiple genes as well as relationship between the cells and the sensitivity to multiple drugs. On the other hand, PLS type 1 (PLS1) enables one to identify important genes for the sensitivity to particular drugs based on, for example, the relationship between the cells and expression of multiple genes as well as the relationship between the cells and the sensitivity to particular drugs. As described in the Examples herein, the present inventors experimentally measured drug sensitivities in vitro and in vivo specifically for cancer cell lines derived from colon cancer, lung cancer, breast cancer, prostate cancer, pancreatic cancer, gastric cancer, neuroblastoma, ovarian cancer, melanoma, bladder cancer, and acute myelocytic leukemia. Further, the expression of 10,000 or more types of genes in the cancer cell lines using DNA microarray was analyzed. Then, they analyzed the expression data and drug sensitivity data of these genes by PLS1, and thus constructed a model by which drug sensitivity can be predicted from the expression of the genes. This technique enabled the inventors to determine the degrees of contribution of the respective genes that were involved in the determination of drug sensitivity by the coefficients for the respective analyzed genes. Thereby, it was possible to select only those groups of genes having high degrees of contribution towards sensitivity.

Further, the present inventors reconstructed the PLS1 model using a group of selected genes with a high degree of contribution towards the determination of sensitivity, thereby developing a system that predicts sensitivity with a high degree of precision using a small number of genes. To achieve this system, first, the present inventors used a sequential method, specifically, the modeling power (MP) method. In the MP method, the greater the MP value (Ψ) of a gene is, the more significant the correlation of the gene is considered to be. The MP value was determined for the expression of each gene, and then genes with higher MP values were selected to greatly reduce the number of genes used in model construction. Thus, the inventors selected only genes that highly contributed towards drug sensitivity and succeeded in constructing a model. The square of the predictive correlation coefficient (Q²) of the constructed PLS1 model was significantly increased.

Furthermore, to further reduce the number of genes, the present inventors reconstructed the model using a systematic method. Specifically, a genetic algorithm (GA), an optimization method that has been used recently in the field of engineering, was used. Using this technique, a thorough search was carried out for a combination of genes in which a statistic in the PLS1 model, Q² value, was maximized and the number of selected genes was minimized. In the GA method, first, an appropriate population was prepared; each member of the population was assessed by using an evaluation function (in this case, a function which maximized the Q² value and minimized the number of selected genes); the members with higher evaluation values were then selected. Next, selected multiple members were subjected to selection, crossover, and mutation to artificially generate new members having high evaluation values. These manipulations were repeated to finally provide a population comprising members having high evaluation values. The use of GA successfully achieved a markedly increased Q² value and the reduction of the number of genes.

Thus, a group of genes with high degrees of contribution towards the determination of drug sensitivity could be selected from the genes on the microarray by the method of the present invention. Further, since the principal component can be converted to the original level of gene expression in the model constructed by PLS1, the model gives the coefficients quantitatively for the expression of respective genes (degrees of contribution), similar to typical multiple regression analysis. The sensitivity prediction was carried out based on the profile of gene expression in specimens with unknown drug sensitivity by using the coefficient values. The calculated predictive values were confirmed to agree well with the degree of sensitivity determined experimentally.

Thus, the present inventors succeeded in the selection of genes with high degrees of contribution towards the determination of drug sensitivity based on the analysis of gene expression data in biological specimens and drug sensitivity data using PLS1, and further, the quantitative prediction of the degree of sensitivity by using the genes. The use of the method of the present invention enables one to select important genes that determine the sensitivity to a drug or any other stimulus. The sensitivity of any specimen can be thus predicted by measuring the expression levels of selected genes. Particularly, when the expression level of a gene identified using the constructed model is measured, the predictive value for the sensitivity can be calculated quantitatively from the value according to the model. The sensitivity prediction method of the present invention is useful, for example, to predict whether a certain drug is effective for a target disease. In addition, the method of the present invention is also useful, for example, to classify unknown specimens based on predictive values for sensitivity. Further, the sensitivity predicted using specimens from patients enables the diagnosis of the disease and the selection of a course of treatment. For example, the effectiveness of a drug treatment for a target disease can be predicted, and thereby, drug selection and optimization of the therapeutic method can be achieved.

Namely, the present invention relates to a method for selecting drug sensitivity-determining genes by using gene expression data, and a method for predicting the drug sensitivity of unknown specimens by using the genes selected. More specifically, the present invention relates to:
[1] a method for constructing a model that predicts sensitivity to a drug based on expression levels of genes, said method comprising the steps of:
(a) obtaining sensitivity data for a biological specimen;
(b) obtaining gene expression data for the biological specimen; and
(c) constructing a model by partial least squares method type 1 using said sensitivity data obtained in step (a) and at least a part of said gene expression data for the biological specimen obtained in step (b), wherein said model can predict the sensitivity of the biological specimen to a specific drug;
[2] the method according to [1], wherein, in the step (c), the model is optimized by constructing a model for each of two or more sets of combinations of genes by the partial least squares method type 1 and by selecting those models in which the number of genes is small and/or those models whose Q² value is high;
[3] the method according to [2], wherein, in the step (c), the model is constructed by computing a parameter that represents a degree of contribution for each of the genes and by selecting the genes that have the greater relative parameter;
[4] the method according to [3], wherein the parameter representing the degree of contribution is a modeling power value (Ψ);
[5] the method according to [2], wherein, in the step (c), the model is constructed by generating different combinations of genes based on a genetic algorithm;
[6] the method according to [1], wherein the sensitivity data comprises in vitro sensitivity data for a biological specimen;
[7] the method according to [1], wherein the sensitivity data comprises animal-experimental sensitivity data for a biological specimen;
[8] the method according to [1], wherein the sensitivity data comprises clinical sensitivity data for a biological specimen;
[9] the method according to [1], wherein the drug is selected from the group consisting of the following farnesyltransferase inhibitors:
a) 6-[1-amino-1-(4-chlorophenyl)-1-(1-methylimidazol-5-yl)methyl]-4-(3-chlorophenyl)-1-methylquinolin-2(1H)-one (Code: R115777);
b) (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile (Code: BMS214662);
c) (+)-(R)-4-[2-[4-(3,10-Dibromo-8-chloro-5,6-dihydro-11H-benzo [5,6] cyclohepta[1,2-b]pyridin-11-yl)piperidin-1-yl]-2-oxoethyl] piperidine-1-carboxamide (Code: SCH66336);
d) 4-[5-[4-(3-Chlorophenyl)-3-oxopiperazin-1-ylmethyl]imidazol-1-ylmethyl] benzonitrile (Code: L778123); and
e) 4-[hydroxy-(3-methyl-3H-imidazole-4-yl)-(5-nitro-7-phenyl-benzofuran-2-yl)-methyl]benzonitrile hydrochloride;
[10] the method according to [1], wherein the drug is selected from the group consisting of the following fluorinated pyrimidines:
a) [1-(3,4-Dihydroxy-5-methyl-tetrahydro-furan-2-yl)-5-fluoro-2-oxo-1,2-dihydro-pyrimidin-4-yl]-carbamic acid butyl ester (Code: capecitabine (Xeloda@));
b)-1-(3,4-Dihydroxy-5-methyl-tetrahydro-furan-2-yl)-5-fluoro-1H-pyrimidine-2,4-dione (Code: Furtulon);
c) 5-Fluoro-1H-pyrimidine-2,4-dione (Code: 5-FU);
d) 5-Fluoro-1-(tetrahydro-2-furanyl)-2,4(1H,3H)-pyrimidinedione (Code: Tegafur);
e) A combination of Tegafur and 2,4(1H,3H)-pyrimidinedione (Code: UFT);
f) A combination of Tegafur, 5-chloro-2,4-dihydroxypyridine and potassium oxonate (molar ratio of 1:0.4:1) (Code: S-1); and
g) 5-Fluoro-N-hexyl-3,4-dihydro-2,4-dioxo-1(2H)-pyrimidinecarboxamide (Code: Carmofur);
[11] the method according to [1], wherein the drug is selected from the group consisting of the following taxanes:
a) [2aR-[2aα,4β,4aβ, 6β,9α(αR*,βS*),11α,12α,12aα,12bα]]-β-(benzoylamino)-α-hydroxybenzenepropanoic acid 6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester (Code: Taxol);
b) [2aR-[2aα,4β,4aβ,6β,9α(αR*,βS*,11α,12α,12aα,12bα)]-β-[[(1,1-dimethylethoxy)carbonyl]amino]-α-hydroxybenzenepropanoic acid 12b-(acetyloxy)-12-(benzoyloxy)-2a,3,4,4a,5,6,9,10,11,12,12a,12b-dodecahydro-4,6,11-trihydroxy-4a,8,13,13-tetramethyl-5-oxo-7,11-methano-1H-cyclodeca [3,4]benz [1,2-b]oxet-9-yl ester (Code: Taxotere);
c) (2R,3S)-3-[[(1,1-dimethylethoxy)carbonyl]amino]-2-hydroxy-5-methyl-4-hexenoic acid (3aS,4R,7R,8aS,9S,10aR,12aS,12bR,13S,13aS)-7,12a-bis(acetyloxy)-13-(benzyloxy)-3a,4,7,8,8a,9,10,10a,12,12a,12b,13-dodecahydro-9-hydroxy-5,8a,14,14-tetramethyl-2,8-dioxo-6,13a-methano-13aH-oxeto[2″,3″:5′,6′]benzo[1′,2′:4,5]cyclodeca[1,2-d]-1,3-dioxol-4-yl ester (Code: IDN 5109);
d) (2R,3S)-β-(benzoylamino)-α-hydroxybenzenepropanoic acid (2aR,4S,4aS,6R,9S,11S,12S,12aR,12bS)-6-(acetyloxy)-12-(benzoyloxy)-2a,3,4,4a,5,6,9,10,11,12,12a,12b-dodecahydro-4,11-dihydroxy-12b-[(methoxycarbonyl)oxy]-4a,8,13,13-tetramethyl-5-oxo-7,11-methano-1H-cyclodeca[3,4]benz[1,2-b]oxet-9-yl ester (Code: BMS 188797); and
e) (2R,3S)-β-(benzoylamino)-α-hydroxybenzenepropanoic acid (2aR,4S,4aS,6R,9S,11S,12S,12aR,12bS)-6,12b-bis(acetyloxy)-12-(benzoyloxy)-2a,3,4,4a,5,6,9,10,11,12,12a,12b-dodecahydro-11-hydroxy-4a,8,13,13-tetramethyl-4-[(methylthio)methoxy]-5-oxo-7,11-methano-1H-cyclodeca[3,4]benz[1,2-b]oxet-9-yl ester (Code: BMS 184476);
[12] the method according to [1], wherein the drug is selected from the group consisting of the following camptothecins:
a) 4(S)-ethyl-4-hydroxy-1H-pyrano[3′,4′:6,7]indolizino[1,2-b]quinoline-3,14(4H,12H)-dione (abbreviation: camptothecin);
b) [1,4′-bipiperidine]-1′-carboxylic acid, (4S)-4,11-diethyl-3,4,12,14-tetrahydro-4-hydroxy-3,14-dioxo-1H-pyrano[3′,4′:6,7]indolizino[1,2-b]quinolin-9-yl ester, monohydrochloride (Code: CPT-11);
c) (4S)-10-[(dimethylamino)methyl]-4-ethyl-4,9-dihydroxy-1H-pyrano[3′:6,7]indolizino[1,2-b]quinoline-3,14(4H,12H)-dione monohydrochloride (abbreviation: Topotecan);
d) (1S,9S)-1-amino-9-ethyl-5-fluoro-9-hydroxy-4-methyl-2,3,9,10,13,15-hexahydro-1H,12H-benzo[de]pyrano[3′,4′:6,7]indolizino[1,2-b]quinoline-10,13-dione (Code: DX-8951f);
e) 5 (R)-ethyl-9,10-difluoro-1,4,5,13-tetrahydro-5-hydroxy-3H,15H-oxepino[3′,4′:6,7]indolizino[1,2-b]quinoline-3,15-dione (Code: BN-80915);
f) (S)-10-amino-4-ethyl-4-hydroxy-1H-pyrano[3′,4′:6,7]indolizino[1,2-b]quinoline-3,14(4H,12H)-dione (Code: 9-aminocamptotecin);
g) 4 (S)-ethyl-4-hydroxy-10-nitro-1H-pyrano[3′,4′:6,7]-indolizino[1,2-b]quinoline-3,14(4H,12H)-dione (Code: 9-nitrocamptothecin);
[13] the method according to [1], wherein the drug is selected from the group consisting of the following nucleoside analogue antitumor drugs:
a) 2′-deoxy-2′,2′-difluorocytidine (Code: DFDC);
b) 2′-deoxy-2′-methylidenecytidine (Code: DMDC);
c) (E)-2′-deoxy-2′-(fluoromethylene) cytidine (Code: FMDC);
d) 1-(β-D-arabinofuranosyl) cytosine (Code: Ara-C);
e) 4-amino-1-(2-deoxy-β-D-erythro-pentofuranosyl)-1,3,5-triazin-2(1H)-one (abbreviation: decitabine);
f) 4-amino-1-[(2S,4S)-2-(hydroxymethyl)-1,3-dioxolan-4-yl]-2(1H)-pyrimidinone (abbreviation: troxacitabine);
g) 2-fluoro-9-(5-O-phosphono-β-D-arabinofuranosyl)-9H-purin-6-amine (abbreviation: troxacitabine); and
h) 2-chloro-2′-deoxyadenosine (abbreviation:
cladribine);
   [14] the method according to [1], wherein the drug is selected from the group consisting of the following dolastatins:
   a) N,N-dimethyl-L-valyl-N-[(1S,2R)-2-methoxy-4[(2S)-2-[(1R,2R)-1-methoxy-2-methyl-3-oxo-3-[[(S)-2-phenyl-1-(2-thiazolyl)ethyl]amino]propyl]-1-pyrrolidinyl]-1-[(is)-1-methylpropyl]-4-oxobutyl]-N-methyl-L-valinamide (abbreviation: dolastatin 10);
   b) cyclo[N-methylalanyl-(2E,4E,10E)-15-hydroxy-7-methoxy-2-methyl-2,4,10-hexadecatrienoyl-L-valyl-N-methyl-L-phenylalanyl-N-methyl-L-valyl-N-methyl-L-valyl-L-prolyl-N2-methylasparaginyl] (abbreviation: dolastatin 14);
   c) (1S)-1-[[(2S)-2,5-dihydro-3-methoxy-5-oxo-2-(phenylmethyl)-1H-pyrrol-1-yl]carbonyl]-2-methylpropyl ester N,N-dimethyl-L-valyl-L-valyl-N-methyl-L-valyl-L-prolyl-L-proline (abbreviation: dolastatin 15);
   d) N,N-dimethyl-L-valyl-N-[(1S,2R)-2-methoxy-4-[(2S)-2-[(1R,2R)-1-methoxy-2-methyl-3-oxo-3-[(2-phenylethyl)amino]propyl]-1-pyrrolidinyl]-1-[(1S)-1-methylpropyl]-4-oxobutyl]-N-methyl-L-valinamide (Code: TZT 1027); and
   e) N,N-dimethyl-L-valyl-L-valyl-N-methyl-L-valyl-L-prolyl-N-(phenylmethyl)-L-prolinamide (abbreviation: cemadotin);
   [15] the method according to (1], wherein the drug is selected from the group consisting of the following anthracyclines:
   a) (8S,10S)-10-[(3-amino-2,3,6-trideoxy-L-lyxo-hexopyranosyl)oxy]-7,8,9,10-tetrahydro-6,8,11-trihydroxy-8-(hydroxyacetyl)-1-methoxynaphthacene-5,12-dione hydrochloride (abbreviation: adriamycin);
   b) (8S,10S)-10-[(3-amino-2,3,6-trideoxy-L-arabino-hexopyranosyl) oxy]-7,8,9,10-tetrahydro-6,8,11-trihydroxy-8-(hydroxyacetyl)-1-methoxynaphthacene-5,12-dione hydrochloride (abbreviation: epirubicin);
   c) 8-acetyl-10-[(3-amino-2,3,6-trideoxy-L-lyxo-hexopyranosyl) oxy]-7,8,9,10-tetrahydro-6,8,11-trihydroxy-1-methoxynaphthacene-5,12-dione, hydrochloride (abbreviation: daunomycin); and
   d) (7S,9S)-9-acetyl-7-[(3-amino-2,3,6-trideoxy-L-lyxo-hexopyranosyl) oxy]-7, 8, 9, 10-tetrahydro-6,9,11-trihydroxynaphthacene-5,12-dione (abbreviation: idarubicin);
   [16] the method according to [1], wherein the drug is selected from the group consisting of the following protein kinase inhibitors:
   a) N-(3-chloro-4-fluorophenyl)-7-methoxy-6-[3-(4-morpholinyl)propoxy]-4-quinazolinamine (Code: ZD 1839);
   b) N-(3-ethynylphenyl)-6,7-bis(2-methoxyethoxy)-4-quinazolinamine (Code: CP 358774);
   c) N⁴-(3-bromophenyl)-N-6-methylpyrido[3,4-d]pyrimidine-4,6-diamine (Code: PD 158780);
   d) N-(3-chloro-4-((3-fluorobenzyl)oxy)phenyl)-6-(5-(((2-methylsulfonyl)ethyl)amino)methyl)-2-furyl)-4-quinazolinamine (Code: GW 2016);
   e) 3-[(3,5-dimethyl-1H-pyrrol-2-yl)methylene]-1,3-dihydro-2H-indol-2-one (Code: SU5416);
   f) (Z)-3-[2,4-dimethyl-5-(2-oxo-1,2-dihydro-indol-3-ylidenemethyl)-1H-pyrrol-3-yl]-propionic acid (Code: SU6668);
   g) N-(4-chlorophenyl)-4-(pyridin-4-ylmethyl)phthalazin-1-amine (Code: PTK787);
   h) (4-bromo-2-fluorophenyl)[6-methoxy-7-(1-methylpiperidin-4-ylmethoxy)quinazolin-4-yl]amine (Code: ZD6474);
   i) N⁴-(3-methyl-1H-indazol-6-yl)-N²-(3,4,5-trimethoxyphenyl)pyrimidine-2,4-diamine (Code: GW2286);
   j) 4-[(4-methyl-1-piperazinyl)methyl]-N-[4-methyl-3-[[4-(3-pyridinyl)-2-pyrimidinyl]amino]phenyl]benzamide (Code: STI-571)
   k) (9α, 10β, 11β, 13α)-N-(2,3,10,12,13-hexahydro-10-methoxy-9-methyl-1-oxo-9,13-epoxy-1H,9H-diindolo[1,2,3-gh:3′,2′,1′-1m]pyrrolo[3,4-j]f1,7]benzodiazonin-11-yl)-N-methylbenzamide (Code: CGP41251);
   l) 2-[(2-chloro-4-iodophenyl)amino]-N-(cyclopropylmethoxy)-3,4-difluorobenzamide (Code: CI1040); and
   m) N-(4-chloro-3-(trifluoromethyl)phenyl)-N′-(4-(2-(N-methylcarbamoyl)-4-pyridyloxy)phenyl)urea (Code: BAY439006);
   [17] the method according to [1], wherein the drug is selected from the group consisting of the following platinum antitumor drugs:
   a) cis-diaminodichloroplatinum(II) (abbreviation: cisplatin);
   b) diammine(1,1-cyclobutanedicarboxylato)platinum(II) (abbreviation: carboplatin); and
   c) hexaamminedichlorobis[μ-(1,6-hexanediamine-κN:κN′)]tri-,stereoisomer,tetranitrate platinum(4+) (Code: BBR3464);
   [18] the method according to [1], wherein the drug is selected from the group consisting of the following epothilones:
   a) 4,8-dihydroxy-5,5,7,9,13-pentamethyl-16-[(1E)-1-methyl-2-(2-methyl-4-thiazolyl)ethenyl]-(4S,7R,8S,9S,13Z,16S)-oxacyclohexadec⁻¹³-ene-2,6-dione (abbreviation: epothilone D);
   b) 7,11-dihydroxy-8,8,10,12,16-pentamethyl-3-[(1E)-1-methyl-2-(2-methyl-4-thiazolyl)ethenyl]-, (1S,3S,7S,10R,11S,12S,16R)-4,17-dioxabicyclo[.14.1.0]heptadecane-5,9-dione6-dione (abbreviation: epothilone); and
   c) (1S,3S,7S,10R,11S,12S,16R)-7,11-dihydroxy-8,8,10,12,16-pentamethyl-3-[(1E)-1-methyl-2-(2-methyl-4-thiazolyl)ethenyl]-17-oxa-4-azabicyclo[14.1.0]heptadecane-5,9-dione (Code: BMS247550);
   [19] the method according to [1], wherein the drug is selected from the group consisting of the following aromatase inhibitors:
   a) α,α,α′,α′-tetramethyl-5-(1H-1,2,4-triazol-1-ylmethyl)-1,3-benzenediacetonitrile (Code: ZD1033);
   b) (6-methyleneandrosta-1,4-diene-3,17-dione (Code: FCE24304); and
   c) 4,4′-(1H-1,2,4-triazol-1-ylmethylene)bis-benzonitrile (Code: CGS20267);
   [20] the method according to [1], wherein the drug is selected from the group consisting of the following hormone modulators:
   a) 2-[4-[(lZ)-1,2-diphenyl-1-butenyl]phenoxy]-N,N-dimethylethanamine (abbreviation: tamoxifen);
   b) [6-hydroxy-2-(4-hydroxyphenyl)benzo[b]thien-3-yl][4-[2-(1-piperidinyl)ethoxy]phenyl]methanone hydrochloride (Code: LY156758);
   c) 2-(4-methoxyphenyl)-3-[4-[2-(1-piperidinyl)ethoxy]phenoxy]benzo[b]thiophene-6-ol hydrochloride (Code: LY353381);
   d) (+)-7-pivaloyloxy-3-(4′-pivaloyloxyphenyl)-4-methyl-2-(4″-(2″1′-piperidinoethoxy)phenyl)-2H-benzopyran (Code: EM800);
   e) (E)-4-[1-[4-[2-(dimethylamino)ethoxy]phenyl]-2-[4-(1-methylethyl)phenyl]-1-butenyl]phenol dihydrogen phosphate(ester) (Code: TAT59);
   f) 17-(acetyloxy)-6-chloro-2-oxapregna-4,6-diene-3,20-dione (Code: TZP4238);
   g) (+,−)-N-[4-cyano-3-(trifluoromethyl)phenyl]-3-[(4-fluorophenyl)sulfonyl]-2-hydroxy-2-methylpropanamide (Code: ZD176334); and
   h) 6-D-leucine-9-(N-ethyl-L-prolinamide)-10-deglycinamide luteinizing hormone-releasing factor (pig) (abbreviation: leuprorelin);
   [21] the method according to [1], wherein the biological specimen is a cancer cell or a cancer cell line;
   [22] the method according to [1], wherein the sensitivity comprises an antitumor effect;
   [23] the method according to [1], wherein the gene expression data comprises high-density nucleic acid array data;
   [24] a method for selecting genes that contribute to biological sensitivity to a high degree, said method comprising the step of selecting part or all of the combinations of genes in a model constructed by the method according to any one of [1] or [2];
   [25] a method for predicting the sensitivity of a test specimen toward a particular stimulus, said method comprising the steps of:
   (a) obtaining, for the test specimen, at least a part of a gene expression data from a model specimen constructed by the method according to [1]; and
   (b) correlating to the fact that the sensitivity is high, a high level of expression of a gene having a positive coefficient in the model and a low level of expression of a gene having a negative coefficient in the model, and correlating to the fact that the sensitivity is low, a low level of expression of a gene having a positive coefficient in the model and a high level of expression of a gene having a negative coefficient in the model;
   [26] the method according to [25], wherein:
   step (a) comprises the step of obtaining the gene expression data in the model for the test specimen; and
   step (b) comprises the step of computing the sensitivity by applying the expression data to the model;
   [27] a computer device that predicts the sensitivity of a test specimen toward a particular stimulus, said device comprising:
   (a) a means for storing a parameter (model coefficient) representing the relationship between gene expression data and sensitivity value in a model constructed by the method according to [1];
   (b) a means for inputting the gene expression data into the model;
   (c) a means for storing the expression data;
   (d) a means for predictively calculating the sensitivity value from the expression data and the parameter (model coefficient) based on the model;
   (e) a means for storing the predictively calculated sensitivity value; and
   (f) a means for outputting the predictively calculated sensitivity value or a result obtained from the sensitivity value;
   [28] a method for producing a high-density nucleic acid array, said method comprising the step of immobilizing or generating, on a support, nucleic acids comprising at least 15 nucleotides comprised in nucleotide sequences encoding respective genes selected by the method according to [24];
   [29] a method for producing a probe or a primer for quantitative or semi-quantitative PCR for respective genes selected by the method according to [24], said method comprising the step of synthesizing nucleic acids comprising at least 15 nucleotides comprised in nucleotide sequences encoding the respective genes; and
   [30] a kit comprising:
   (a) a high-density nucleic acid array, or a probe or a primer for quantitative or semi-quantitative PCR, wherein said array, probe, or primer comprises nucleic acids comprising at least 15 nucleotides from nucleotide sequences encoding respective genes selected by the method according to [24]; and
   (b) a storage medium which records the sensitivity to drugs predicted using the array, or the probe or the primer.

A report by Okamura et al. relating to factors determining the sensitivity to drugs or irradiation describes a method for estimating genes that greatly contribute towards the sensitivity based on a simple regression analysis of gene expression and sensitivity (Okamura et al. (2000) Int. J. Oncol. 16:295-303). This method is based on simple regression analysis, but it is difficult to uniquely select only a specific, significant group of genes with this method, because gene expression is correlative. Accordingly, in general, this method cannot be applied to analyze the relationship between multiple gene expression and sensitivity.

Musumarra et al. have reported a method for selecting a group of genes commonly exhibiting a strong correlation between compounds that act by the same mechanism, using Soft Independent Modeling of Class Analogy (SIMCA) (Musumarra et al. (2001) J. Comp. -Aid. Mol. Design 15:219-234). Hilsenbeck et al. have also reported identification of resistance-determining factors for particular drugs using principal component analysis (PCA) (Hilsenbeck et al. (1999) J. Natl. Cancer Inst. 91:453-459). These methods are based on the principal component analysis, and therefore allow merely the selection of genes that greatly contribute towards sensitivity, but are not useful to quantitatively predict drug sensitivity. Using the multivariate analysis technique (PLS type 2) (Musumarra et al. (2001) Biochem. Pharma. 62: 547-553), Musumarra et al. have also reported the selection of a group of genes exhibiting strong correlations common to the effect of a group of compounds sharing common mechanism of action. However, with this method, it is difficult to estimate a group of genes that greatly contribute towards sensitivity to a particular drug and to predict the sensitivity towards other unknown specimens. The method of the present invention enables one to construct a model to quantitatively predict the sensitivity to a desired particular drug based on gene expression data. The present invention is particularly useful to construct a system for predicting sensitivity based on the determined correlation between the sensitivity to a particular drug and high-density nucleic acid array data.

According to the method of the present invention, a model is constructed based on the analysis of the correlation between the sensitivity to a particular drug and gene expression data using PLS1. The term “a model is constructed” by PLS1 analysis means obtaining an equation representing the relationship between the sensitivity value and the principal component obtained from gene expression data by PLS1 analysis. Since the principal component can be converted to the original level of gene expression, the coefficients for the respective gene expression (degrees of contribution) can be estimated quantitatively. With these coefficient values, the sensitivity can be predicted from the gene expression profiles for sensitivity-unknown specimens. Further, with the model provided by PLS1 analysis, it is possible to determine the square of the correlation coefficient (R²) and the square of the predictive correlation coefficient (Q²). These statistics are discussed later.

As used herein, the term “sensitivity” to a drug means the responsiveness of a biological specimen towards the drug, in other words, the effect the drug has on the specimen. The use of the method of the present invention enables the construction of a model that allows the prediction of the sensitivity to a desired drug. The present invention is particularly useful to construct a model for predicting the antitumor effect as the sensitivity, in which the antitumor effect can be predicted using anti-tumor drugs or other drug candidate compounds. The antitumor effect specifically includes the effect of suppressing tumor cell growth, the effect of suppressing tumor growth, activity of inducing tumor cell death, etc. The term “degree of contribution” of a gene for determining the sensitivity means the degree of correlation between the gene expression and sensitivity.

The term “biological specimen” means a specimen obtained from an organism, including cells, tissues, organs, etc. In constructing a model for predicting the above-mentioned antitumor effect, cancer cells or cancer cell lines are preferably used as biological specimens. For constructing a model that allows the prediction of an antitumor effect of a particular drug on a wide variety of cancers, it is preferable to construct the model using data obtained by using cancer cells or cancer cell lines derived from various cancers. For example, it is preferable to obtain drug sensitivity data and gene expression data using biological specimens including cells or cell lines of at least two or more types, preferably five or more types, more preferably seven or more types, most preferably ten or more types of cancers selected from the group consisting of: colon cancer, lung cancer, breast cancer, prostate cancer, pancreatic cancer, gastric cancer, neuroblastoma, ovarian cancer, melanoma, bladder cancer, acute myelocytic leukemia, uterine cancer, endometrial cancer, and liver cancer. There are many known cancer cell lines derived from the above cancers, for example, HCT116 (ATCC CCL-247), WiDr (ATCC CCL-218), COLO201 (ATCC CCL-224), COLO205 (ATCC CCL-222), COLO320DM (ATCC CCL-220), LoVo (ATCC CCL-229), HT-29 (ATCC HTB-38), DLD-1 (ATCC CCL-221), SW480 (ATCC CCL-228), LS411N (ATCC CRL-2159), LS513 (ATCC CRL-2134), HCT15 (ATCC CCL-225), and CX-1 (Japanese Foundation for Cancer Research, Japan; Division of Cancer Treatment, Tumor Repository, NCI. Osieka, R., Johnson, R. K. Evaluation of chemical agents in phase I clinical trial and earlier stages of development against xenografts of human colon carcinoma. Editor(s): Houchens, D. P. & Ovejera, A. A. Proc. Symp. Use Athymic (Nude) Mice Cancer Res. 1978. 217-23.) (all of the above are colon cancer cell lines); QG56 (purchased from Immuno-Biological Laboratories Co., Ltd., Japan (IBL)), Calu-1 (ATCC HTB-54), Calu-3 (ATCC HTB-55), Calu-6 (ATCC HTB-56), PC1 (purchased from Immuno-Biological Laboratories Co., Ltd., Japan), PC10 (purchased from Immuno-Biological Laboratories Co., Ltd., Japan), PC13 (purchased from Immuno-Biological Laboratories Co., Ltd., Japan), NCI-H292 (ATCC CRL-1848), NCI-H441 (ATCC HTB-174), NCI-H460(ATCC HTB-177), NCI-H596 (ATCC HTB-178), PC14 (The Institute of Physical and Chemical Research (RIKEN), Japan. RCB0446; IBL), NCI-H69 (ATCC HTB-119), LXFL529 (Dr. H. H. Fiebig, Freiburg Univ., Germany, Berger, D. P., Fiebig, H. H., Winterhalter, B. R. Establishment and characterization of human tumor xenograft models in nude mice. In Fiebig, H. H. and Berger, D. P., eds. Immunodeficient Mice in Oncology. Basel, Karger, 1992,23-46.), LX-1 (Japanese Foundation for Cancer Research, Japan; Division of Cancer Treatment, Tumor Repository, NCI. Houchens, D. P., Ovejera, A. A. and Barker, A. D.; and The therapy of human tumors in athymic (nude) mice. Proc. Symp. Use Athymic (Nude) Mice Cancer Res. 1978. 267-80.), and A549 (ATCC CCL-185) (all of the above are lung cancer cell lines); MDA-MB-231 (ATCC HTB-26), MDA-MB-435S (ATCC HTB-129), T-47D (ATCC HTB-133), Hs578T (ATCC HTB-126), MCF7 (ATCC HTB-22), ZR-75-1 (ATCC CRL-1500), MAXF401 (Dr. H. H. Fiebig, Freiburg Univ, Germany, Berger, D. P., Fiebig, H. H., Winterhalter, B. R. Establishment and characterization of human tumor xenograft models in nude mice. In Fiebig, H. H. and Berger, D. P., eds. Immunodeficient Mice in Oncology. Basel, Karger, 1992,23-46.), and MX1 (Japanese Foundation for Cancer Research, Japan; Division of Cancer Treatment, Tumor Repository, NCI. Ovejera, A. A., Houchens. D. P. and Barker A. D. Chemotherapy of human tumor xenografts in genetically athymic mice. Ann. Clin. Lab. Sci. 1978. 8: 50-56.) (all of the above are breast cancer cell lines); PC-3 (ATCC CRL-1435), DU145 (ATCC HTB-81), and LNCaP-FGC (ATCC CRL-1740) (all of the above are prostate cancer cell lines); AsPC-1 (ATCC CRL-1682), Capan-1 (ATCC HTB-79), Capan-2 (ATCC HTB-80), BxPC3 (ATCC CRL-1500), PANC-1 (ATCC CRL-1469), Hs766T (ATCC HTB-134), MIA PaCa-2 (ATCC CRL-1420), and SU.86.86 (ATCC CRL-1834) (all of the above are pancreatic cancer cell lines); MKN-45 (purchased from Immuno-Biological Laboratories Co., Ltd., Japan), MKN28 (purchased from Immuno-Biological Laboratories Co., Ltd., Japan), and GXF97 (Dr. H. H. Fiebig, Freiburg Univ., Germany, Berger, D. P., Fiebig, H. H., Winterhalter, B. R. Establishment and characterization of human tumor xenograft models in nude mice. In Fiebig, H. H. and Berger, D. P., eds. Immunodeficient Mice in Oncology. Basel, Karger, 1992,23-46.) (gastric cancer cell line); T98G (ATCC CRL-1690) (neuroblastoma cell line); IGROV1 (through The Netherlands Cancer Institute, Netherland, Benard, J., Da Silva, J., De Blois, M-C., Boyer, P., Duvillard, P., Chiric, E. and Riou, G. Characterization of a human ovarian adenocarcinoma line, IGROV1, in tissue culture and in nude mice. Cancer Res. 1985 45: 4970-4979), SK-OV-3 (ATCC HTB-77), and Nakajima (Faculty of Medicine, Niigata University, Yanase, T., Tamura, M., Fujita, K., Kodama, S., Tanaka, K. Inhibitory effect of angiogenesis inhibitor TNP-470 on tumor growth and metastasis of human cell lines in vitro and in vivo. Cancer Res. 1993. 53: 2566-2570.) (ovarian cancer cell line); C32 (ATCC CRL-1585) (melanoma cell line); HT-1197 (ATCC CRL-1437), T24 (ATCC HTB-4), and Scaber (ATCC HTB-3) (bladder cancer cell line); KG-1a (ATCC CCL-246.1) (cell line of acute myelocytic leukemia); Yumoto (Chiba Cancer Center, Tokita, H., Tanaka, N., Sekimoto, K., Ueno, T., Okamoto, K. and Fujimura, S. Experimental model for combination chemotherapy with metronidazole using human uterine cervical carcinomas transplanted into nude mice. Cancer Res. 1980 40: 4287-4294.) (uterine cancer cell line); ME-180 (ATCC HTB-33) (endometrial cancer cell line); HepG2 (ATCC HB-8065), Huh-1 (Japanese Collection of Research Bioresources, Japan. JCRB0199), Huh7 (Japanese Collection of Research Bioresources, Japan (JCRB), JCRB0403), and PLC/PRF/5 (ATCC CRL-8024) (liver cancer cell line); and KB (ATCC CCL-17) (oral epithelial cancer). An excellent model for predicting the sensitivity of a wide variety of cancers can be constructed by obtaining the drug sensitivity data and gene expression data using biological specimens including at least five or more types, preferably ten or more types, more preferably fifteen or more types, most preferably twenty or more types of cell lines selected from the group consisting of these cancer cell lines and by carrying out model construction according to the present invention. Further, for constructing a sensitivity prediction system for a particular type of cancer, it is preferable to construct the model using cells from the target type of cancer.

Drug sensitivity data of biological specimens are obtained for the model construction of the present invention. The sensitivity data may be in vitro data or in vivo data. Further, there is no limitation on the type of data; such data may be quantitative data consisting of continuous or discrete values. The sensitivity data consisting of continuous values are preferably, for example, ICso for drugs, tumor growth inhibition rate (TGI %), blood level of tumor markers, etc. The tumor growth inhibition rate that can be measured, for example, using a xenograft model for cancer cells and can be used as in vivo drug sensitivity data. Specifically, for example, a cancer cell mass is subcutaneously transplanted in a mouse, and then a drug is administered in vivo to determine the effect of suppressing the growth of the transplanted tumor (TGI %).

The sensitivity data consisting of discrete values are preferably data categorized by the degree of sensitivity, etc. Such categorization is achieved, for example, by preparing some classification criteria depending on the degree of drug sensitivity and then by classifying the biological specimens according to the criteria. As described above, not only continuous quantitative values but also discrete data can be used in the present invention. By using categorization, qualitative sensitivity data can be quantified. Thus, arbitrary data reflecting the degree of sensitivity can be used in the present invention.

In the present invention, there is no limitation on the type of drug for which the sensitivity is predicted. It is possible to use desired drugs that act on biological specimens (cells, tissue, and so forth.). The present invention is useful to construct a model for predicting the sensitivity to particularly pharmaceuticals or candidate compounds thereof, by using them or compositions comprising them. Particularly, anti-tumor drugs, candidate compounds thereof, or the like can be suitably used.

Such drugs preferably include, for example, farnesyltransferase inhibitors, specifically including 6-[1-amino-1-(4-chlorophenyl)-1-(1-methylimidazol-5-yl)methyl]-4-(3-chlorophenyl)-1-methylquinolin-2(1H)-one (Code: R115777), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile (Code: BMS214662), (+)-(R)-4-[2-[4-(3,10-Dibromo-8-chloro-5,6-dihydro-11H-benzo[5,6]cyclohepta[1,2-b]pyridin-11-yl)piperidin-1-yl]-2-oxoethyl]piperidine-1-carboxamide (Code: SCH66336), 4-[5-[4-(3-Chlorophenyl)-3-oxopiperazin-1-ylmethyl]imidazol-1-ylmethyl]benzonitrile (Code: L778123), and 4-[hydroxy-(3-methyl-3H-imidazole-4-yl)-(5-nitro-7-phenyl-benzofuran-2-yl)-methyl]benzonitrile hydrochloride. The preferable drugs also include, for example, pyrimidine fluorides, specifically including [1-(3,4-Dihydroxy-5-methyl-tetrahydro-furan-2-yl)-5-fluoro-2-oxo-1,2-dihydro-pyrimidin-4-yl]-carbamic acid butyl ester (Code: capecitabine (Xeloda®)), 1-(3,4-Dihydroxy-5-methyl-tetrahydro-furan-2-yl)-5-fluoro-1H-pyrimidine-2,4-dione (Code: Furtulon), 5-Fluoro-1H-pyrimidine-2,4-dione (Code: 5-FU), 5-Fluoro-1-(tetrahydro-2-furanyl)-2,4(1H,3H)-pyrimidinedione (Code: Tegafur), a combination of Tegafur and 2,4(1H,3H)-pyrimidinedione (Code: UFT), a combination of Tegafur, 5-chloro-2,4-dihydroxypyridine and potassium oxonate (molar ratio of 1:0.4:1) (Code: S-1), and 5-Fluoro-N-hexyl-3,4-dihydro-2,4-dioxo-1 (2H)-pyrimidinecarboxamide (Code: Carmofur). Other preferable drugs are, for example, taxanes, specifically including [2aR-[2aα,4β,4aβ,6β,9α(αR*,βS*),11α,12α,12aα,12bα]]-β-(benzoylamino)-α-hydroxybenzenepropanoic acid 6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca[3,4]benz [1,2-b]oxet-9-yl ester (Code: Taxol), [2aR-[2aα,4β,4aα, 6β,9α(αR*,βS*,11α,12α,12aα,12bα)]-β-[[(1,1-dimethylethoxy)carbonyl]amino]-α-hydroxybenzenepropanoic acid 12b-(acetyloxy)-12-(benzoyloxy)-2a,3,4,4a,5,6,9,10,11,12,12a,12b-dodecahydro-4,6,11-trihydroxy-4a,8,13′,13-tetramethyl-5-oxo-7,11-methano-1H-cyclodeca[3,4]benz[1,2-b]oxet-9-yl ester (Code: Taxotere), (2R,3S)-3-[[(1,1-dimethylethoxy)carbonyl]amino]-2-hydroxy-5-methyl-4-hexenoic acid(3aS,4R,7R,8aS,9S,10aR,12aS,12bR,13S,13aS)-7,12a-bis(acetyloxy)-13-(benzyloxy)-3a,4,7,8,8a,9,10,10a,12,12a,12b,13-dodecahydro-9-hydroxy-5,8a,14,14-tetramethyl-2,8-dioxo-6,13a-methano-13aH-oxeto[2″,3″:5′,6′]benzo[1′,2′:4,5]cyclodeca[1,2-d]-1,3-dioxol-4-yl ester (Code: IDN 5109), (2R,3S)-β-(benzoylamino)-α-hydroxy benzenepropanoic acid(2aR,4S,4aS,6R,9S,11S,12S,12aR,12bS)-6-(acetyloxy)-12-(benzoyloxy)-2a,3,4,4a,5,6,9,10,11,12,12a,12b-dodecahydro-4,11-dihydroxy-12b-[(methoxycarbonyl)oxy]-4a,8,13,13-tetramethyl-5-oxo-7,11-methano-1H-cyclodeca[3,4]benz[1,2-b]oxet-9-yl ester (Code: BMS 188797), and (2R,3S)-β-(benzoylamino)-α-hydroxy benzenepropanoic acid(2aR,4S,4aS,6R,9S,11S,12S,12aR,12bS)-6,12b-bis(acetyloxy)-12-(benzoyloxy)-2a,3,4,4a,5,6,9,10,11,12,12a,12b-dodecahydro-11-hydroxy-4a,8,13,13-tetramethyl-4-[(methylthio)methoxy]-5-oxo-7,11-methano-1H-cyclodeca[3,4]benz[1,2-b]oxet-9-yl ester (Code: BMS 184476). The preferable drugs also include, for example, camptothecins, specifically including 4(S)-ethyl-4-hydroxy-1H-pyrano[3′,4′:6,7]indolizino[1,2-b]quinoline-3,14(4H,12H)-dione (abbreviation: camptothecin), [1,4′-bipiperidine]-1′-carboxylic acid, (4S)-4,11-diethyl-3,4,12,14-tetrahydro-4-hydroxy-3,14-dioxo-1H-pyrano[3′,4′:6,7]indolizino[1,2-b]quinolin-9-yl ester, monohydrochloride (Code: CPT-11), (4S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride (abbreviation: Topotecan), (1S,9S)-1-amino-9-ethyl-5-fluoro-9-hydroxy-4-methyl-2,3,9,10,13,15-hexahydro-1H,12H-benzo(de]pyrano[3′,4′:6,7]indolizino[1,2-b]quinoline-10,13-dione (Code: DX-8951f), 5(R)-ethyl-9,10-difluoro-1,4,5,13-tetrahydro-5-hydroxy-3H,15H-oxepino[3′,4′:6,7]indolizino[1,2-b]quinoline-3,15-dione (Code: BN-80915), (S)-10-amino-4-ethyl-4-hydroxy-1H-pyrano[3′,4′:6,7]indolizino[1,2-b]quinoline-3,14(4H,12H)-dione (Code: 9-aminocamptotecin), 4(S)-ethyl-4-hydroxy-10-nitro-1H-pyrano[3′,4′:6,7]-indolizino[1,2-b]quinoline-3,14 (4H,12H)-dione (Code: 9-nitrocamptothecin), The preferable drugs also include, for example, nucleoside analogue antitumor drugs, specifically including 2′-deoxy-21,2′-difluorocytidine (Code: DFDC), 2′-deoxy-2′-methylidenecytidine (Code: DMDC), (E)-2′-deoxy-2′-(fluoromethylene)cytidine (Code: FMDC), 1-(β-D-arabinofuranosyl)cytosine (Code: Ara-C), 4-amino-1-(2-deoxy-β-D-erythro-pentofuranosyl)-1,3,5-triazin-2(1H)-one (abbreviation: decitabine), 4-amino-1-[(2S,4S)-2-(hydroxymethyl)-1,3-dioxolan-4-yl]-2(1H)-pyrimidinone (abbreviation: troxacitabine), 2-fluoro-9-(5-O-phosphono-β-D-arabinofuranosyl)-9H-purin-6-amine (abbreviation: troxacitabine), 2-chloro-2′-deoxyadenosine (abbreviation: cladribine). The preferred drugs also include, for example, dolastatins, specifically including N,N-dimethyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-L-valinamide (abbreviation: dolastatin 10), cyclo[N-methylalanyl-(2E,4E,10E)-15-hydroxy-7-methoxy-2-methyl-2,4,10-hexadecatrienoyl-L-valyl-N-methyl-L-phenylalanyl-N-methyl-L-valyl-N-methyl-L-valyl-L-prolyl-N2-methylasparaginyl] (abbreviation: dolastatin 14), (1S)-1-[[(2S)-2,5-dihydro-3-methoxy-5-oxo-2-(phenylmethyl)-1H-pyrrol-1-yl]carbonyl]-2-methylpropyl ester N,N-dimethyl-L-valyl-L-valyl-N-methyl-L-valyl-L-prolyl-L-proline (abbreviation: dolastatin 15), N,N-dimethyl-L-valyl-N-[(1S,2R)-2-methoxy-4-[(2S)-2-[(1R,2R)-1-methoxy-2-methyl-3-oxo-3-[(2-phenylethyl)amino]propyl]-1-pyrrolidinyl]-1-[(1S)-1-methylpropyl]-4-oxobutyl]-N-methyl-L-valinamide (Code: TZT 1027), and N,N-dimethyl-L-valyl-L-valyl-N-methyl-L-valyl-L-prolyl-N-(phenylmethyl)-L-prolinamide (abbreviation: cemadotin) The preferred drugs also include, for example, anthracyclines, specifically including (8S,10S)-10-[(3-amino-2,3,6-trideoxy-L-lyxo-hexopyranosyl)oxy]-7,8,9,10-tetrahydro-6,8,11-trihydroxy-8-(hydroxyacetyl)-1-methoxynaphthacene-5,12-dione hydrochloride (abbreviation: adriamycin), (8S,10S)-10-[(3-amino-2,3,6-trideoxy-L-arabino-hexopyranosyl)oxy]-7,8,9,10-tetrahydro-6,8,11-trihydroxy-8-(hydroxyacetyl)-1-methoxynaphthacene-5,12-dione hydrochloride (abbreviation: epirucicin), 8-acetyl-10-[(3-amino-2,3,6-trideoxy-L-lyxo-hexopyranosyl)oxy]-7,8,9,10-tetrahydro-6,8,11-trihydroxy-1-methoxynaphthacene-5,12-dione, hydrochloride (abbreviation: daunomycin), and (7S,9S)-9-acetyl-7-[(3-amino-2,3,6-trideoxy-L-lyxo-hexopyranosyl)oxy]-7,8,9,10-tetrahydro-6,9,11-trihydroxy-naphthacene-5,12-dione (abbreviation: idarubicin). The preferred drugs also include, for example, protein kinase inhibitors, specifically including N-(3-chloro-4-fluorophenyl)-7-methoxy-6-(3-(4-morpholinyl)propoxy]-4-quinazolinamine (Code: ZD 1839), N-(3-ethynylphenyl)-6,7-bis(2-methoxyethoxy)-4-quinazolinamine (Code: CP 358774), N⁴-(3-bromophenyl)-N-6-methylpyrido [3,4-d]pyrimidine-4,6-diamine (Code: PD 158780), N-(3-chloro-4-((3-fluorobenzyl)oxy)phenyl)-6-(5-(((2-methylsulfonyl)ethyl)amino)methyl)-2-furyl]-4-quinazolinamine (Code: GW 2016), 3-[(3,5-dimethyl-1H-pyrrol-2-yl)methylene]-1,3-dihydro-2H-indol-2-one (Code: SU5416), (Z)-3-[2,4-dimethyl-5-(2-oxo-1,2-dihydro-indol-3-ylidenemethyl)-1H-pyrrol-3-yl]-propionic acid (Code: SU6668), N-(4-chlorophenyl)-4-(pyridin-4-ylmethyl) phthalazin-1-amine (Code: PTK787), (4-bromo-2-fluorophenyl) [6-methoxy-7-(1-methyl-piperidin-4-ylmethoxy)quinazolin-4-yl]amine (Code: ZD6474), N⁴-(3-methyl-1H-indazol-6-yl)-N²-(3,4,5-trimethoxy-phenyl)pyrimidine-2,4-diamine (Code: GW2286), 4-[(4-methyl-1-piperazinyl)methyl]-N-[4-methyl-3-[[4-(3-pyridinyl)-2-pyrimidinyl]amino]phenyl]benzamide (Code: STI-571), (9α,10β,11β,13α)-N-(2,3,10,12,13-hexahydro-10-methoxy-9-methyl-1-oxo-9,13-epoxy-1H,9H-diindolo[1,2,3-gh:3′,2′,1′-1m]pyrrolo[3,4-j][1,7]benzodiazonin-11-yl)-N-methylbenzamide (Code: CGP41251), 2-[(2-chloro-4-iodophenyl)amino]-N-(cyclopropylmethoxy)-3,4-difluorobenzamide (Code: CI1040), and N-(4-chloro-3-(trifluoromethyl)phenyl)-N′-(4-(2-(N-methylcarbamoyl)-4-pyridyloxy)phenyl)urea (Code: BAY439006). Further, the preferred drugs include, for example, platinum antitumor drugs, specifically including cis-diaminodichloroplatinum(II)(abbreviation: cisplatin), diammine(1,1-cyclobutanedicarboxylato) platinum(II) (abbreviation: carboplatin), and hexaamminedichlorobis[μ-(1,6-hexanediamine-κN:κN′)]tri-,stereoisomer,tetranitrate platinum(4+) (Code: BBR3464). The preferable drugs also include epothilones, specifically including 4,8-dihydroxy-5,5,7,9,13-pentamethyl-16-[(1E)-1-methyl-2-(2-methyl-4-thiazolyl)ethenyl]-(4S,7R,8S,9S,13Z,16S)-oxacyclohexadec-13-ene-2,6-dione (abbreviation: epothilone D), 7,11-dihydroxy-8,8,10,12,16-pentamethyl-3-[(1E)-1-methyl-2-(2-methyl-4-thiazolyl)ethenyl]-, (1S,3S,7S,10R,11S,12S,16R)-4,17-dioxabicyclo [14.1.0]heptadecane-5,9-dione6-dione (abbreviation: epothilone), and (1S,3S,7S,10R,11S,12S,16R)-7,11-dihydroxy-8,8,10,12,16-pentamethyl-3-[(1E)-1-methyl-2-(2-methyl-4-thiazolyl)ethenyl]-17-oxa-4-azabicyclo[14.1.0]heptadecane-5,9-dione (Code: BMS247550). The preferable drugs also include aromatase inhibitors, specifically including α,α,α′,α′-tetramethyl-5-(1H-1,2,4-triazol-1-ylmethyl)-1,3-benzenediacetonitrile (Code: ZD1033), (6-methyleneandrosta-1,4-diene-3,17-dione (Code: FCE24304), and 4,4′-(1H-1,2,4-triazol-1-ylmethylene)bis-benzonitrile (Code: CGS20267). The preferred drugs also include hormone modulators, for example, including 2-[4-[(1Z)-1,2-diphenyl-1-butenyl]phenoxy]-N,N-dimethylethanamine (abbreviation: tamoxifen), [6-hydroxy-2-(4-hydroxyphenyl)benzo[blthien-3-yl](4-[2-(1-piperidinyl)ethoxy]phenyl]methanone hydrochloride (Code: LY156758), 2-(4-methoxyphenyl)-3-[4-[2-(1-piperidinyl)ethoxy]phenoxy]benzo[b]thiophene-6-ol hydrochloride (Code: LY353381), (+)-7-pivaloyloxy-3-(4′-pivaloyloxyphenyl)-4-methyl-2-(4″′(2″′-piperidinoethoxy)phenyl)-2H-benzopyran (Code: EM800), (E)-4-[1-[4-[2-(dimethylamino)ethoxy]phenyl]-2-[4-(1-methylethyl)phenyl]-1-butenyl]phenol dihydrogen phosphate(ester) (Code: TAT59), 17-(acetyloxy)-6-chloro-2-oxapregna-4,6-diene-3,20-dione (Code: TZP4238), (+,−)-N-[4-cyano-3-(trifluoromethyl)phenyl]-3-[(4-fluorophenyl)sulfonyl]-2-hydroxy-2-methylpropanamide (Code: ZD176334), and 6-D-leucine-9-(N-ethyl-L-prolinamide)-10-deglycinamide luteinizing hormone-releasing factor (pig) (abbreviation: leuprorelin).

In the model construction of the present invention, gene expression data are obtained from biological specimens for which drug sensitivity data have been obtained. In addition to the same specimens for which drug sensitivity data were obtained, gene expression data may be obtained from other specimens as well, for example, for other specimen aliquots simultaneously collected or for specimens derived from the same origin. For example, when the gene expression profile of an established cell line has been determined previously, drug sensitivity data can be obtained from the established cell line obtained separately and can be applied to the method of the present invention using the expression profile. The model construction of the present invention is achieved by using expression data of at least two or more genes, preferably five or more genes, more preferably ten or more genes, even more preferably twenty or more (for example, thirty or more, forty or more, or fifty or more) genes.

Gene expression data can be obtained by any method, for example, by a method for determining RNA levels, such as Northern hybridization, and quantitative or semi-quantitative RT (reverse transcription)-PCR, or a method for determining protein levels, such as ELISA (enzyme linked immunosorbent assay) and Western blotting. Preferably, the measurement is carried out with a method by which a great amount of gene expression data can be extensively obtained. Such a method includes an analysis using high-density nucleic acid array. “The high-density nucleic acid array” means a substrate on which many nucleic acids have been bound in a small area. The nucleic acid may be DNA or RNA, which may include artificial or modified nucleotides. The substrate is typically made of glass, but may be made of nylon, nitrocellulose, or other types of resins. In general, a DNA-bound high-density nucleic acid array is also called a DNA microarray. “A high-density nucleic acid array” refers to an array to which nucleic acid molecules are bound at a density of typically about 60 or higher per 1 cm², more preferably about 100 or higher, even more preferably about 600 or higher, even more preferably about 1,000, about 5,000, about 10,000, or about 40,000 or higher, most preferably about 100,000 or higher. There is no limitation on the length of the nucleic acid molecule; the nucleic acid can be a relatively long polynucleotide such as a cDNA or a fragment thereof, or an oligonucleotide. The length of nucleic acids bound to the substrate typically ranges from 100 to 4000 nucleotides, preferably from 200 to 4000 nucleotides, for a cDNA; or ranges from 15 to 500 nucleotides, preferably from 30 to 200 nucleotides, even more preferably from 50 to 200 nucleotides, for an oligonucleotide. Arrays are particularly suitable for the present invention because owing to the small surface area of an array, the hybridization conditions for the respective probes (nucleic acids on the array) are highly homogeneous, and also a very large number of probes can hybridize simultaneously. When gene expression data obtained with a high-density nucleic acid array are used for model construction, the expression data used typically comprise data for 100 or more genes, more preferably 500 or more, even more preferably 1000 or more (for example, 2000 or more, 5000 or more, or 10000 or more) genes. The genes suitable for the model construction can be selected from many genes.

The gene expression data may be obtained in the absence or presence of a drug.

Further, gene expression data may be obtained in vitro or in vivo. In vivo expression date can be obtained, for example, by rapidly freezing biological specimens taken out from an individual in liquid nitrogen, and extracting RNAs by a known method. The prediction of the physiologically relevant sensitivity can be achieved based on the model of the present invention constructed by the combined use of the in vivo gene expression data and in vivo drug sensitivity data.

Based on the drug sensitivity data and gene expression data obtained as described above, the model is constructed by the partial least squares method type 1. The number of sensitivity data used for the analysis (the number of biological specimens used for model construction) is at least two or more, preferably ten or more, more preferably fifteen or more, most preferably twenty or more. The correlation between the antitumor effect of a particular drug and high-density nucleic acid array data can be revealed by analyzing the data according to the present invention. The important gene(s) can be estimated quantitatively based on the gene expression coefficient for each gene (the degree of contribution) obtained by the analysis. Further, the antitumor effect can be predicted from gene expression data of unknown specimens by using the gene expression coefficient for each gene obtained by the analysis.

In constructing the model, it is preferable to select data from a large number of gene expression data. Genes used for data analysis can be selected, for example, by pre-treating high-density nucleic acid array data as follows.

i) Pre-Treatment of Data

After Fold Change (FC) values of test specimens are calculated relative to the standard specimen for all the genes, it is preferable to use those genes that have relatively high standard deviations of FC used for the analysis, and those that are expressed in most specimens used in the analysis. For example, genes having standard deviations of FC equal to 2 or more and whose expression was found in 25% or more of the entire number of specimens used for the analysis may be used.

When a GeneChip from Affymetrix is used, the FC value relative to the standard value for each specimen is calculated according to Affymetrix® Microarray Suite User Guide (p3⁵8) based on the following equation:

$${FC}_{k} = ( \frac{{AvgDiffChange}_{k}}{max [ min ( {AvgDiff}_{exp , k} , {AvgDiff}_{base , k} ) , 2.8 * {Q}_{c} ]} ) + ( \begin{matrix}+ 1 if {AvgDif f}_{exp , k} > {AvgDiff}_{base , k} \\ - 1 if {AvgDif f}_{exp , k} < {AvgDiff}_{base , k}\end{matrix} )$$

$$Where {Q}_{c} = max ( {Q}_{exp} , {Q}_{base} )$$

$$AvgDiff Change = {AvgDif f}_{exp , k} - {AvgDiff}_{base , k}$$

In the equation, FCₖ represents FC value of gene k; AvgDiff_{exp,k} represents the expression level of gene k in a test specimen; AvgDiff_{base,k} represents the expression level of gene k in the standard specimen; Q represents the background (noise) of the measured value in each experiment; and Qₑₓₚ and Q_{base} represent the Q values for the test specimen and standard specimen, respectively.
ii) Statistical Treatment

Partial least squares method type 1 (PLS1) (Geladi et al. (1986) Anal. Chim. Acta 185: 1-17) is used as the statistical method. PLS1 analysis can be carried out on a computer. The software for the analysis can be prepared according to the algorithm described in the above-mentioned reference.

As necessary, the gene expression data and drug sensitivity data can be converted to any data format suitable for statistical treatment. Such conversion includes, for example, standardization and logarithmic conversion. For example, when gene expression is assayed with a DNA microarray, it is preferable to use Xᵢₖ−\overline{X}ᵢ (Xᵢₖ represents FC value of gene k for specimen i; \overline{X}ᵢ represents average FC value of a selected gene of specimen i) as the expression data of gene k for specimen i. In addition, when IC₅₀ is used as the sensitivity data, it is preferable to statistically treat the data using log(1/IC₅₀).

Performance evaluations of PLS model can be conducted by using two indices, the square of the correlation coefficient, R², and the square of the predictive correlation coefficient, Q².

The square of the correlation coefficient R² and the square of the predictive correlation coefficient Q² are defined as follows:
R²=1−S1/S2
S1=Σ(yᵢ−ŷ)²
S2=Σ(yᵢ−\overline{y})²
where \overline{y} and ŷᵢ represent the average of y (antitumor effect) and computed value of yᵢ in the model equation, respectively, and yi represents the sensitivity value for specimen i.
Q²=1−S1′/S2′
S1′=Σ(yᵢ−y_{i,pred})²
S2′=Σ(yᵢ−\overline{y})²
where \overline{y} and Y_{i,pred} represent the average of y (antitumor effect) and the value of yᵢ predicted in the model equation by the leave-one-out method, respectively. In the leave-one-out method, the model is constructed from all but one specimen, and the predictive y value of the specimen that was left out is obtained. This procedure is repeated to determine the predictive values for all the specimens.

In general, Q² value is more frequently used than R² value to evaluate model performance. Namely, as Q² value is nearer to 1.0, the model is more predictive for an unknown specimen.

iii) Model Optimization by Gene Selection

It is preferable to construct the model by using the minimum number of genes selected from an available gene pool. Thereby, the amount of gene expression data that is required for sensitivity prediction can be reduced and the degree of predictability (Q²) can be improved. The present invention provides a method of model optimization, in which the above model is constructed by conducting the partial least squares method type 1 for each combination of two or more sets of genes and model optimization is achieved by selecting a model with the smallest number of genes and/or highest Q² value. It is preferable to select genes with high degrees of contribution towards drug sensitivity. Such a selection can be achieved by any desired method. For example, model construction can be carried out by using all the genes at the first step, followed by selecting the genes with relatively high absolute values of coefficients (the degrees of contribution) More preferred selection methods include the method using modeling power (MP).

Since modeling power (Ψ value) is an index representing the degrees of contribution of each gene towards drug sensitivity, it can be assumed that, the gene having the greater value has a more important meaning in explaining drug sensitivity.
Ψₖ=1−Sₖ/S_{k,x}
Sₖ=[Σ(yᵢₖ−ŷᵢₖ)²/(n−A−1)]^{1/2}
S_{k,x}=[Σ(Xᵢₖ−\overline{X}ₖ)²/(n−1)]^{1/2}
where n represents the number of specimens; A represents the number of components in PLS1; ŷᵢₖ represents the computed value for the antitumor effect on specimen i when only the k-th gene is used. \overline{X}ₖ represents the average Fc value of expression data of the k-th gene, and Xᵢₖ represents expression data of gene k in specimen i.

For example, the model can be constructed by selecting only the genes having an MP value (Ψₖ) greater than a particular value (cut-off value) and using the expression data of these genes to construct the model. The cut-off value may be determined, for example, so as to select about half, 25%, or 10% of the entire number of genes, but is not limited thereto. For example, in the Example herein, the present inventors reduced the number of genes by selecting genes having MP value greater than 0.3, or greater than 0.1, and thus succeeded in increasing the degree of predictability (Q²) of the model. In this way, the model of the present invention can be optimized by carrying out gene selection using MP.

It is also preferable to conduct gene selection by a systematic method. For example, instead of selecting genes with high degrees of contribution, genes are pre-selected by an alternative method to construct a model by using the genes, and then gene selection can be carried out by identifying combinations of genes by which a more optimized model is constructed. Such a method includes the method using the genetic algorithm (GA).

The genetic algorithm is an optimization method that is being used recently in the field of engineering. For example, this technique enables one to thoroughly search combinations of genes for maximized Q² value, which is a statistic in the PLS1 model, and for a minimized number of selected genes. According to the genetic algorithm, first, an appropriate population is prepared, every member in the population is assessed by using an evaluation function (in this case, a function which maximizes the Q² value and minimizes the number of selected genes), and members with higher evaluation values are then selected. Next, through selection, crossover, and mutation, the multiple members selected are artificially converted to novel members having higher evaluation values. These manipulations are repeated to finally produce a population comprising members having higher evaluation values. The genetic algorithm can be performed by a computer using an executable program prepared according to literature (Rogers et al. (1994) J. Chem. Inf Comput. Sci. 34: 854-866).

For the specific evaluation function, for example, the following defining equation is preferably used:
Evaluation function=Q²−α*K
where Q² represents the square of the predictive correlation coefficient in the PLS1 model; K represents the number of selected genes; α represents an appropriate penalty value.

Further, the present invention relates to a method for selecting genes having high degrees of contribution towards the determination of the drug sensitivity, comprising the step of selecting a part of or the entire combinations of genes in the model constructed as described above. For example, for selecting a part of genes from the combinations of genes in the model, it is preferable to select genes having a high degree of contribution towards the sensitivity. To achieve this selection, for example, genes with relatively greater absolute values of coefficients in the model can be selected. The greater the coefficient, the stronger the correlation to sensitivity is. When the coefficient is positive, the correlation is also positive, thus, the higher the gene expression level, the higher the sensitivity. When the coefficient is negative, the correlation is also negative, thus, the higher the gene expression level, the lower the sensitivity. There is no limitation on the number of genes selected; for example, top-1, 5, 10, 15, 20, 50, or 100 genes having high absolute coefficient values can be selected.

Further, it is also preferable to select all the combinations genes used for model construction. Highly accurate predictive sensitivity values can be obtained by applying the expression data of selected genes to the model. Further, for example, when the number of genes to be selected or the upper limit is previously determined, the number of genes or the upper limit can be fixed and the evaluation function for the above GA can be determined so as to maximize the Q² value. By this treatment, an optimized model can be constructed with the determined number of genes.

The selected genes are useful to predict the degree of drug sensitivity of a biological specimen of interest. In addition, these genes can be candidates for target genes for the drug, and thus can be targeted for drug development. Further, the genes may be useful as disease markers, and thus may enable the assessment of the progress of a disease or the treatment status by monitoring the expression of the marker genes.

iv) Prediction of the Antitumor Effect

The sensitivity prediction can be achieved by measuring the expression levels of genes selected in test specimens according to PLS1 model construction or the gene selection technique. The present invention provides a method for predicting the sensitivity of a test specimen toward a particular stimulus, said method comprising the steps of: (a) obtaining, for the test specimen, at least a part of a gene expression data from a model specimen constructed by the method of the present invention; and (b) correlating to the fact that the sensitivity is high, a high level of expression of a gene having a positive coefficient in the model and a low level of expression of a gene having a negative coefficient in the model, and correlating to the fact that the sensitivity is low, a low level of expression of a gene having a positive coefficient in the model and a high level of expression of a gene having a negative coefficient in the model. The method of the present invention enables the qualitative or quantitative prediction, and particularly, is useful to quantitatively predict the sensitivity. As used herein, the term “quantitative” prediction means the prediction of the degree of sensitivity by at least three categories or more, preferably four or more, more preferably five or more, even more preferably six or more, and most preferably, it is predicted sequentially. For example, the quantitative prediction includes when the sensitivity is predicted as a sequential value, and when at least three or more discrete categories classified based on the degree of sensitivity are predicted.

As described above, a positive coefficient represents a positive correlation with sensitivity, and a negative one represents a negative correlation. Thus, a test specimen is tested for the expression of genes having a positive coefficient and/or the expression of genes having a negative coefficient. When the expression level of a gene having a positive coefficient is relatively higher than that in other specimens and/or when the expression level of a gene having the negative coefficient is relatively lower than that in other specimens, the test specimen is assessed to have high drug sensitivity. Alternatively, when the expression level of a gene having the positive coefficient is relatively lower than that in other specimens and/or when the expression level of a gene having the negative coefficient is relatively higher in the test specimen as compared with that in other test specimens, the test specimen is assessed to have low drug sensitivity. When the expression of multiple genes is tested, it is preferable to put weight on the expression data having higher absolute coefficient values. For example, placing weight depending on the absolute coefficient value allows a more accurate prediction of quantitative sensitivity.

Most preferably, the method of the present invention for predicting the sensitivity is a method, in which: step (a) comprises the step of obtaining the gene expression data in the model for the test specimen; and step (b) comprises the step of computing the sensitivity by applying the expression data to the model. Namely, the present invention provides a method for predicting the sensitivity of a test specimen, comprising the steps of: (a) obtaining, for the test specimen, all gene expression data of a model constructed by the method of the present invention; and (b) computing, based on the model, the sensitivity value from a parameter (model coefficient) representing the correlation between gene expression data and the sensitivity value of the model. The computed value for the drug sensitivity can be obtained based on the coefficient for each gene according to the following equation:
Calculated activity for i=Σ(coefficientₖ×(Xᵢₖ−\overline{X}ᵢ)+\overline{y})
where coefficientₖ represents a coefficient for gene k; Xᵢₖ represents a FC value of gene k in specimen i; \overline{X}ᵢ represents the average FC value of the selected gene in specimen i; and \overline{y} represents the average of y (antitumor effect).

The predictive value of sensitivity computed based on the above equation quantitatively indicates the degree of predictability. Alternatively, it is possible to achieve the prediction in which the sensitivity is assessed to be positive when the predictive value is higher than a particular value or assessed to be negative when it is identical to or lower than the value. Such a threshold can be determined by experimentally measuring the drug sensitivity. Further, the sensitivity can be categorically estimated by using a constant assign to a range according to the sensitivity. For example, the TGI % allows the categorization as shown in Example herein. Thus, the method of prediction of the present invention comprises not only obtaining a predictive sensitivity value that can be computed based on the above equation but also deriving a secondary result from the predictive sensitivity value.

Biological specimens can be classified based on the result of sensitivity prediction as described above. This method comprises the steps of: (a) assaying test biological specimens for the expression level of a gene selected by the method of the present invention; (b) predicting the drug sensitivity from the gene expression data according to the method of the present invention; and (c) classifying the biological specimens based on the prediction. For example, based on the predictive sensitivity value, the test specimens can be classified into sensitive and non-sensitive groups, or alternatively into smaller groups according to the degree of sensitivity. Further, the degree of sensitivity of the test specimen may reflect not only drug sensitivity, but also differences in other characteristics, and thus, the classification method can be effective in various types of classifications.

In addition, a disease can be diagnosed based on the result of the prediction of the sensitivity carried out by using test specimens from diseased individuals. This method comprises the steps of: (a) assaying test biological specimens obtained from diseased individuals for the expression level of a gene selected by the method of the present invention; (b) predicting the drug sensitivity from the gene expression data according to the method of the present invention; and (c) diagnosing the disease based on the prediction. In addition to the classification described above, this method allows the diagnosis of whether the disease of the subject is sensitive or insensitive to the drug, or the diagnosis of the degree of sensitivity. The prediction of the sensitivity to respective candidate therapeutic drug allows the assessment of the most effective and thus the selection of a suitable therapy for the disease.

For example, in one embodiment, the method comprises deciding whether the drug is to be administered or not, or estimating the dose of the drug, based on the predictive drug sensitivity value computed according to the above method of the present invention. For example, when the predictive value of sensitivity to a particular drug, which has been computed according to the above method, is high, then the drug can be administered. On the other hand, when the predictive sensitivity value computed is low, then the drug is not administered or alternatively can be used in combination with other therapeutic methods. Such a therapeutic selection is useful to optimize the therapy for each disease type or to select therapeutic methods suitable for each patient even when there are patients who have been affected with the same disease.

For example, for a disease of a certain patient, when the predictive drug sensitivity value computed by the above method is high, then the drug can be administered. On the other hand, when the predictive sensitivity value computed is low, then the drug is not administered or alternatively can be used in combination with other therapeutic methods. Further, drug sensitivity can be judged collectively in combination with results of other tests or diagnoses. So far, The uniform medical care that does not take differences between individuals into consideration, so-called ready-made health care, was carried out. The above method of the present invention allows precise sensitivity prediction based on the differences in the levels of gene expression between different diseases or between individuals, and thereby allows precise selection of therapeutics, prescription including dosage, and therapeutic methods. As a result, it is expected that treatments with enhanced effects for each patient, or those with reduced side effects (tailor-made health care) would be implemented.

The sensitivity prediction of the present invention can be achieved by using a computer. For example, the sensitivity is predicted from the gene expression data using a relationship equation of the gene expression level (derived from the model) and the sensitivity using a computer, and then the result is displayed. Namely, the present invention provides a computer device to predict the sensitivity of a test specimen, comprising:
(a) a means for storing a parameter (model coefficient) representing the correlation between gene expression data and sensitivity value of the model constructed by the method as described above;
(b) a means for inputting the gene expression data into the model;
(c) a means for storing the expression data;
(d) a means for predictively calculating the sensitivity value from the expression data and the parameter (model coefficient) based on the model;
(e) a means for storing the predictively calculated sensitivity value; and
(f) a means for outputting the predictively calculated sensitivity value or a result obtained from the sensitivity value.

The above-mentioned “parameter” (model coefficient) means a constant in the relationship equation of gene expression derived from the model constructed by PLS1, specifically, coefficientsₖ (coefficients for gene k) in the following equation to be used for the prediction of the sensitivity of specimen i:
Calculated activity for i=Σ(coefficientₖ×(Xᵢₖ\overline{X}ᵢ)+\overline{y})

Furthermore, the present invention relates to a computer program to carry out the above method of the present invention for predicting the sensitivity. This computer program is used to compute predictive values of the sensitivity to a particular drug from the gene expression data. Further, the present invention provides computer-readable storage media where the above computer program is stored. There is no limitation on the type of storage medium of the present invention as long as it is computer-readable, including both portable and stationary ones. For example, the storage media include CD-ROMs, flexible disks (FD), MOs, DVDS, hard disks, semiconductor memories, etc. The program as described above can be stored in a portable storage medium to be sold, or can be stored in a storage device of a computer which is attached through a network to be transferred to another computer via the network.

In a preferable embodiment, the above computer device of the present invention contains an executable program for conducting the sensitivity predicting method in an auxiliary storage device such as a hard disk. The computer device may further contain another program for controlling the executable program for conducting the method for predicting the sensitivity.

An example of the conformation of the computer device of the present invention is shown in FIG. 7. In the device, input means 1, output means 2, memory 6, and central processing unit (CPU) 3 are integrated connected to one another via bus line 5. The memory 6 contains various programs for conducting the treatments (tasks) of the present invention; parameters required for the computation are also stored therein. The central processing unit (CPU) 3 calculates various data according to the commands provided by these programs. These programs include a program for the predictive calculation of drug sensitivity based on gene expression data and the above parameters, and another program for controlling the program. These programs may contain programs to process the result obtained by the predictive calculation to image data, or programs to classify the specimens or to select candidates for the therapeutic method based on the predictive value. These programs can be combined into one. The gene expression data are fed into the computer by the input means 1. The gene expression data can be transferred into the computer from a portable storage medium, stationary medium such as a hard disk, or communication network such as the Internet, via a receive means such as a modem, in addition to being fed directly into the device of the present invention by an input means such as a keyboard. The input data can be stored in the main memory or temporary storage means 4 of the computer. The central processing unit (CPU) 3 performs predictive calculation of the sensitivity, based on the input expression data according to the commands provided by the above-mentioned program(s). The computed predictive sensitivity value is stored in a storage means or temporary storage means in the computer, and then directly provided as an output via an output means, or provided as an output after being processed by a program to display the result based on the value. This output means comprises output to a storage medium, communication medium, display monitor, printer, etc.

The computer device of the present invention can be connected to a communication medium. Thus, the device can receive gene expression data via online communication, and return the predictive sensitivity value. For example, it is possible to connect the computer device to the Internet so as to carry out the sensitivity prediction online via a web browser.

The present invention also provides a method for preparing probes or primers for quantitative or semi-quantitative PCR for the respective genes, comprising the step of synthesizing nucleic acids comprising at least 15 consecutive nucleotides from nucleotide sequences encoding the respective genes selected by the method of the present invention for selecting genes that highly contribute towards the determination of the above mentioned drug sensitivity. The nucleic acids can be synthesized by a known method such as the phosphoamidite method. The produced probes or primers are useful for assaying the gene expression level in the model construction or sensitivity prediction of the present invention.

The present invention also provides a method for producing a high-density nucleic acid array, comprising the step of immobilizing or generating, on a support, nucleic acids comprising at least 15 consecutive nucleotides from nucleotide sequences encoding the respective genes selected by the method of the present invention for selecting genes that highly contribute towards the determination of the above-mentioned drug sensitivity. Previously known methods for producing high-density nucleic acid array include methods for polymerizing nucleotides on a substrate and for binding polynucleotides to a substrate, and any of these methods can be utilized in the present invention. The produced high-density nucleic acid array is useful for assaying the gene expression level in the model construction or sensitivity prediction of the present invention.

The above-mentioned probes or primers, or high-density nucleic acid array can be provided as a kit for predicting the drug sensitivity. The present invention provides a kit containing: (a) the above-mentioned probes or primers, or high-density nucleic acid array; and (b) a storage medium which records information that sensitivity to drugs can be predicted using them. Such storage media include portable storage media such as paper, CD-ROMs, and flexible disks. Further, the kit of the present invention also includes a kit comprising, for example, an instruction for referring, via a communication medium, another storage medium that has a record that that sensitivity to drugs can be predicted using this kit.

### BRIEF DESCRIPTION OF THE DRAWINGS

FIG. 1 shows the in vitro sensitivity of each cancer cell line to the drug 4-[Hydroxy-(3-methyl-3H-imidazol-4-yl)-(5-nitro-7-phenyl-benzofuran-2-yl)-methyl]benzonitrile hydrochloride. The concentration for inhibiting the cell proliferation to 50% (IC₅₀ value) was determined and presented by log₁₀(1/IC₅₀).

FIG. 2 indicates the in vivo drug sensitivity of each cancer cell line. The tumor growth inhibition rate (TGI %) in the xenograft model is shown.

FIG. 3 shows a result of IC₅₀ prediction based on gene expression data for the test cancer cell lines, according to the PLS1 model constructed from the in vitro gene expression data and in vitro drug sensitivity data for each cancer cell line. The graph indicates computed predictive IC₅₀ values and the actual experimentally determined values. Closed circle represents the cancer cells (learning specimens) used for the model construction; open circle represents cancer cells (test specimens) that were not used for the model construction.

FIG. 4 shows a result of TGI % prediction based on gene expression data for the test cancer cell lines, according to the PLS1 model constructed from the in vivo gene expression data and in vivo drug sensitivity data for each cancer cell line (TGI % value in the xenograft model). The graph indicates computed predictive TGI % values and the actual experimentally determined values. Closed circle represents the cancer cells (learning specimens) used for the model construction; open circle represents cancer cells (test specimens) that were not used for the model construction.

FIG. 5 shows the drug sensitivity of cancer cells categorized based on the in vivo drug sensitivity of each cancer cell line to Xeloda® (TGI % value in the xenograft model).

FIG. 6 shows a result of drug sensitivity prediction of the test cancer cells according to the PLS1 model constructed based on the categorized sensitivity data. The graph indicates the computed predictive score for the sensitivity (computed value) and sensitivity scores categorized based on the actual experimentally determined TGI %. Closed circle represents the cancer cells (learning specimens) used for the model construction; open circle represents cancer cells (test specimens) that were not used for the model construction.

FIG. 7 shows an exemplary structural diagram of a computer device used for predictive computation of drug sensitivity based on gene expression data.

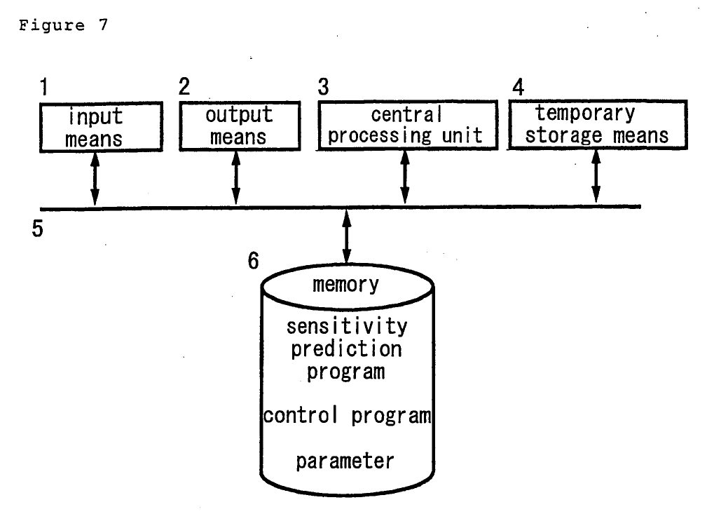

### BEST MODE FOR CARRYING OUT THE INVENTION

The present invention is specifically illustrated below with reference to Examples, but it is not to be construed as being limited thereto. All of the publications cited herein are incorporated by reference in their entirety.

### EXAMPLE 1

### Analysis and Prediction of the Antitumor Effect In Vitro or in the Xenograft Model for 4-[Hydroxy-(3-methyl-3H-imidazol-4-yl)-(5-nitro-7-phenyl-benzofuran-2-yl)-methyl]benzonitrile Hydrochloride

Drug Sensitivity Test

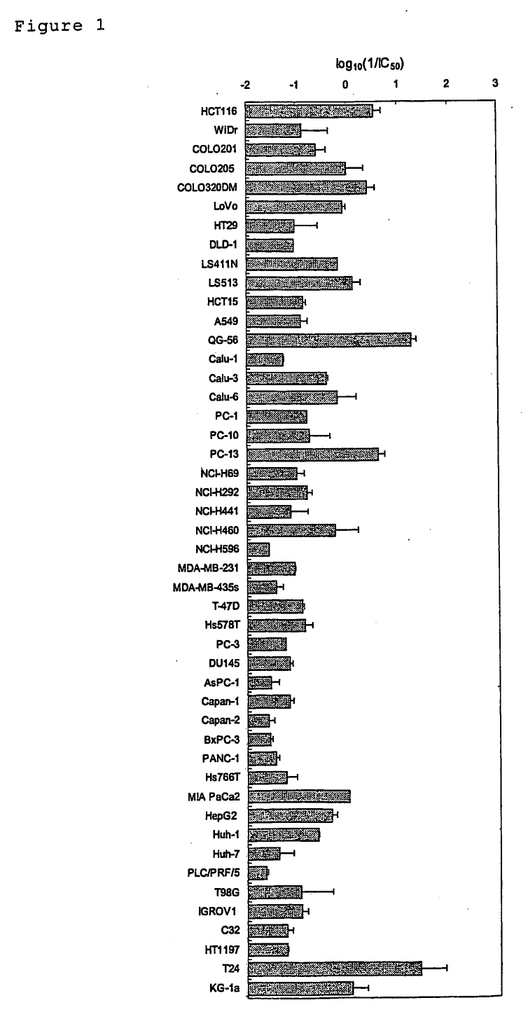

The in vitro drug sensitivity test was carried out with a cell proliferation assay in a micro-titer plate using the MST-8 colorimetric method. The human cancer cells used were HCT116, WiDr, COLO201, COLO205, COLO320DM, LoVo, HT29, DLD-1, LS411N, LS513, and HCT15 (all of the above are colon cancer cell lines); A549, QG56, Calu-1, Calu-3, Calu-6, PC1, PC10, PC13, NCI-H292, NCI-H441, NCI-H460, NCI-H596, and NCI-H69 (all of the above are lung cancer cell lines); MDA-MB-231, MDA-MB-435S, T-47D, and Hs578T (all of the above are breast cancer cell lines); PC-3, and DU145 (all of the above are prostate cancer cell lines); AsPC-1, Capan-1, Capan-2, BxPC3, PANC-1, Hs766T, and MIAPaCa2 (all of the above are pancreatic cancer cell lines); HepG2, Huh1, Huh7, and PLC/PRF/5 (all of the above are hepatic cancer cell lines); T98G (neuroblastoma cell line); IGROV1 (ovarian cancer cell line); C32 (melanoma cell line); HT-1197 and T24 (bladder cancer cell line); and KG-1a (acute myelocytic leukemic cell line). The cells were cultured according to standard methods recommended by ATCC. For example, the cells of colon cancer cell line HCT116 were plated at a cell density of 2,000 cells/well in a 96-well plate, in the presence of the above-mentioned drug in 200 μl MaCoy's medium containing 10% fetal calf serum and cultured at 37° C. in an atmosphere of 5% CO₂ for four days. The IC₅₀ values for the respective cells are shown in FIG. 1.

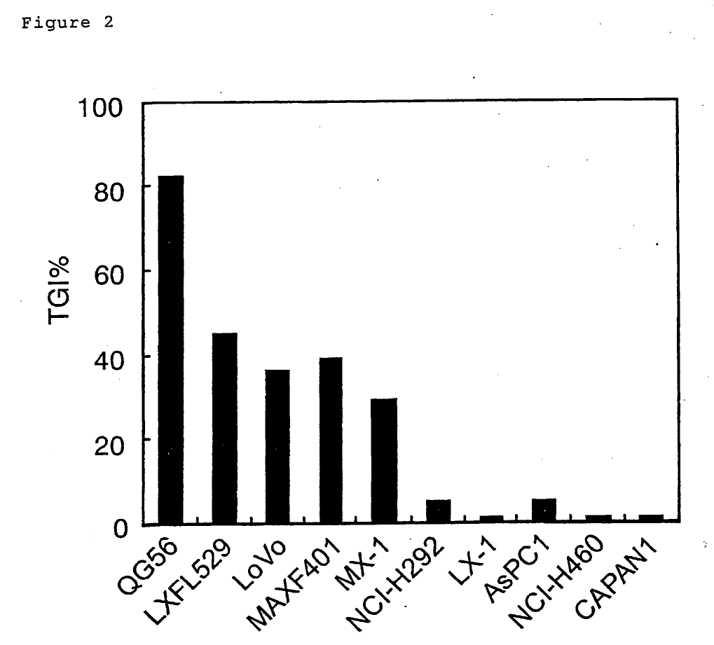

The in vivo sensitivity test was carried out with a Balb/c nu/nu mouse (nude mouse) model in which human cancer cells have been subcutaneous transplanted (xenograft model) Fifteen cell lines were used. Namely HCT116, LoVo, and COLO320DM (all of the above are colon cancer cell lines); LXFL529, LX-1, NCI-H292, NCI-H460, PC13, PC10 and QG56 (all of the above are cell lines of non-small-cell lung cancer); AsPCl and Capan-1 (all of the above are pancreatic cancer cell lines); MAXF401 and MX1 (all of the above are breast cancer cell lines); and C32 (melanoma cell line). 2×10⁶ cells (in 0.2 ml of Hank's solution at a cell density of 1×10⁷ cells/ml) were subcutaneously transplanted to nude mice. After the tumors were allowed to grow to a volume of 300-500 mm, tumor mass were resected and cut into small pieces (3×2×1 mm). Using a trochar, a single tumor piece was subcutaneously transplanted to each mouse in a group of six 6-week old mice. From the third day after transplantation, the drug (200 mg/kg) was orally administered five times a week for two weeks. Based on the average tumor volume on the fourteenth day of administration, the tumor growth inhibition rate (TGI %) relative to that of the untreated group was determined as the in vivo sensitivity (FIG. 2).

Gene Expression Analysis

Gene expression analysis was carried out by using a GeneChip U95A human array from Affymetrix. The in vitro expression was analyzed using the respective cells grown to be sub-confluent in a 75-cm² culture bottle containing the same medium (drug-free) as used in the drug sensitivity test. The total RNA was obtained as follows. The medium was removed from the bottle, and then 1 ml of Sepazol (Nacalai Tesque) was directly added to the bottle to lyze the cells. The cell lysate was transferred to a 15-ml tube, and further mixed to ensure the complete lysis of the cells. 0.2ml of chloroform was added and mixed with the lysate, and then the aqueous layer was separated from the organic layer by centrifugation. The upper aqueous layer was transferred into another tube. After an equal volume of isopropanol was added and mixed with the aqueous layer, RNA was recovered by centrifugation. For testing the in vivo expression, 2×10⁶ cells of each cell line were subcutaneously transplanted into each nude mouse. After the tumors were allowed to grow to a volume of 500-800 mm³, the tumor tissues were cut off from subcutaneous tissues and rapidly frozen in liquid nitrogen. The frozen tumor tissues were ground in liquid nitrogen, mixed with 20 ml Sepasol per 1 g tissue, and vigorously mixed to lyze the cells. 0.2 ml chloroform per 1 ml Sepasol was added to the mixture, and vigorously mixed. Then, the upper aqueous layer was separated from the organic layer by centrifugation, and transferred into an another tube. An equal volume of isopropanol was added and mixed with the aqueous layer, and then total RNA was recovered by centrifugation. The synthesis of complementary DNA, synthesis of complementary RNA by in vitro transcription using T7 RNA polymerase, hybridization, washing, and signal amplification using an antibody were carried out according to the protocols from Affymetrix (GeneChip Technical Manual) The data obtained were normalized by the global scaling method with the target fluorescence intensity at 300 by using Microarray Suite 4.0 software from Affymetrix. FC (Fold Change) value relative to the standard value for each specimen was computed as described above according to Microarray Suite User Guide from Affymetrix (Affymetrix® Microarray Suite User Guide, p358).

Firstly, the in vitro IC₅₀ was used as the sensitivity data. In the analysis of in vitro specimens, the standard data were determined by averaging the values for 23 cell lines: HCT116, WiDr, COLO205, COLO320DM, LoVo, DLD-1, HCT15, Calu-6, NCI-H460, QG56, AsPC-1, Capan1, MDA-MB-231, MDA-MB-435S, T47D, PC-3, DU145, LNCap-FGC, HepG2, Huh7, PLC/PRF/5, T98G, and KG-1a. In the analysis of in vivo specimens, the standard data were determined by averaging the values for 10 cell lines: LoVo, LXFL529, LX-1, NCI-H292, NCI-H460, QG56, AsPC1, Capan-1, MAXF401, and MX1.

Statistical Treatment

The correlation between in vitro gene expression data and in vitro drug sensitivity data (log(1/IC₅₀)) was analyzed by the partial least squares method type 1 (PLS1).

In the pre-treatment of gene expression data, as described above, the FC of a test specimen relative to the standard specimen was computed for every gene. Then, genes having standard deviations of FC equal to 2 or more and whose expression was found in 25% or more of the entire number of specimens used for the analysis were selected. By the pre-treatment, 1,784 genes were selected from the entire 12,559 genes. The correlation between the expression data and drug sensitivity data (log(1/IC₅₀)) for the selected 1,784 genes was assessed by PLS1 (see the above section “ii) statistical treatment”). The PLS1 analysis software was prepared in C language according to the algorithm in a published report (Geladi et al. (1986) Anal. Chim. Acta 185: 1-17).

The treatment resulted in a model consisting of five components, in which the square of the correlation coefficient (R²) was 0.99 and the square of the predictive correlation coefficient (Q²) was 0.32. The modeling power was computed for every gene, and then genes with a value greater than 0.3 were selected as important genes. The modeling power value was computed according to the published report shown in “statistical treatment”. The PLS1 analysis was carried out again by using the expression data of the selected 152 and drug sensitivity data (log(1/IC₅₀)), which resulted in a model consisting of five components, in which the square of the correlation coefficient (R²) was 0.93 and the square of the predictive correlation coefficient (Q²) was 0.39. The value of standard deviations was 0.27. The square of the predictive correlation coefficient (Q²) was revealed to improve by a simple gene selection such as the modeling power. A model consisting of 152 genes was taken as the final model.

Sensitivity Prediction

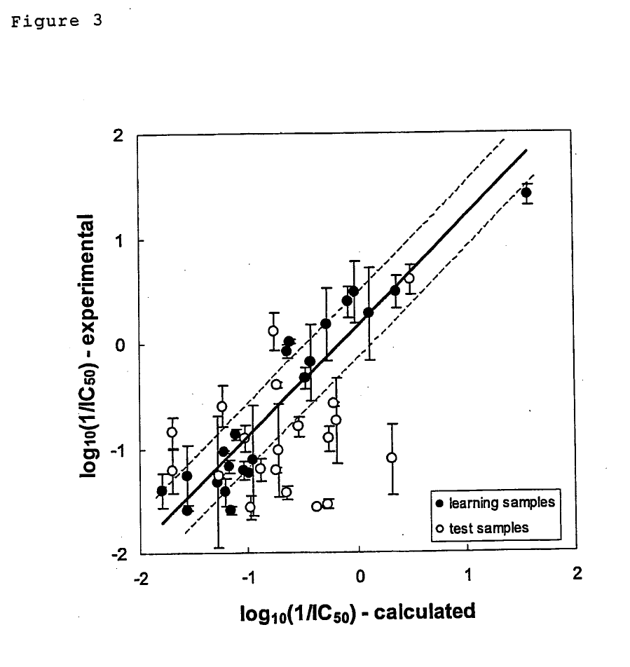

Representative genes selected as above are shown in Table 1. The coefficient corresponds to the degree of correlation—the greater the absolute value, the stronger the correlation. The higher the expression level of a gene having a positive coefficient is, the higher the sensitivity will be. On the other hand, the higher the expression level of a gene having a negative coefficient is, the lower the sensitivity will be. As shown in Table 1, the sensitivity level can be predicted based on the expression levels of selected genes having greater absolute values of the coefficient. Further, the predictive sensitivity value can be computed from the coefficient of the respective genes by applying to the model the expression data for all the genes used in the model construction. A theoretical IC₅₀ was computed from the expression data of 152 genes identified in the final model and the coefficient determined by PLS1, and then compared to the experimental value (FIG. 3). The theoretical value of IC₅₀ was computed based on the coefficient for each gene according to the following equation:
Calculated activity for i=Σ(coefficientₖ×(Xᵢₖ−\overline{X}ᵢ)+\overline{y})
where coefficientₖ represents the coefficient for gene k; Xᵢₖ represents FC value for gene k in specimen i; \overline{X}ᵢ represents the average FC value for a selected gene in specimen i; \overline{y} represents the average of y (antitumor effect).

A theoretical IC₅₀ was determined from the gene expression data of the cell lines, which had not been used in the statistical analysis, by using this model, and then compared to the experimental value. The result showed that the predictability was excellent, and thus this technique was demonstrated to be effective (FIG. 3).

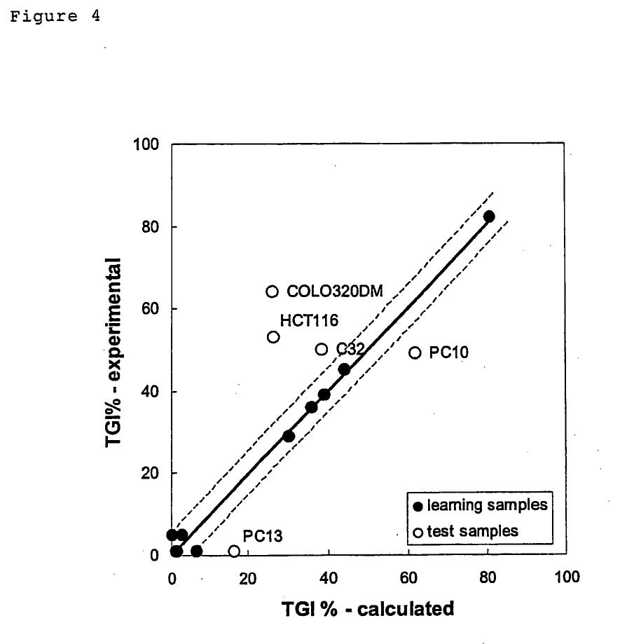

Further, the expression level of every gene belonging to the group of identified 152 genes in xenograft tissues and the antitumor activity in the xenograft model, i.e. TGI %, were analyzed again by PLS1 (R²=0.99, Q²=0.65, SD=3.87). Then, the coefficient was newly computed for each gene. A theoretical TGI % was computed based on this coefficient and gene expression data in the xenograft tissues, and then compared to the experimental value (FIG. 4). By using this model, a theoretical TGI % was determined from gene expression data of various xenograft tissues, having unknown drug sensitivity. A therapeutic experiment was carried out with the xenograft models for HCT116, C32, COLO320DM, PC10, and PC13. The comparison between the resulting experimental value and the theoretical TGI % revealed that the predictability was effective (FIG. 4).

TABLE 1

| GenBank | | |
|---|---|---|
| Ac. No. | Coefficient | Description |
| M16279 | −0.0172 | Antigen identified by mAb 12E7, F21 and O13 |
| X76180 | 0.0158 | sodium channel, nonvoltage-gated 1 alpha |
| M20560 | −0.0154 | annexin A3 |
| U17077 | 0.0149 | BENE protein |
| X78947 | −0.0148 | connective tissue growth factor |
| AI445461 | −0.0144 | similar to transmembrane 4 super family |
| | | member 1 |
| M76125 | −0.0117 | AXL recepror tyrosine kinase |
| AL034374 | 0.0113 | homologue of yeast long chain polyunsaturated |
| | | fatty acid elongation enzyme 2 |
| Y11307 | −0.0111 | cystein rich angiogenic inducer, 61 |

### EXAMPLE 2

### Analysis and Prediction of the Antitumor Effect for Xeloda® in the Xenograft Model for Sensitivity-Unknown Cell Lines (Categorization Model)

Drug Sensitivity Test

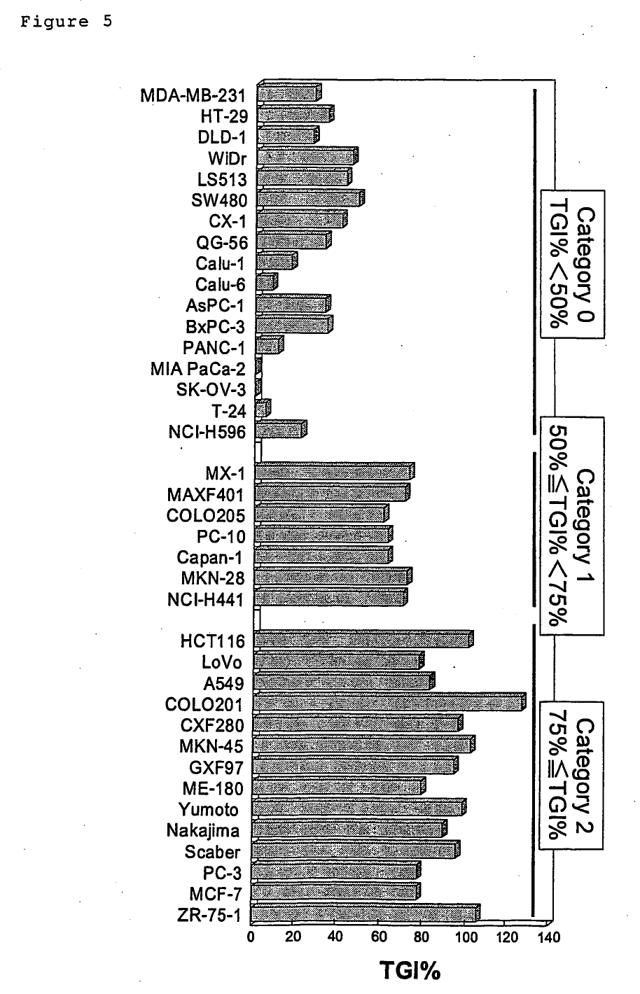

The antitumor effect of Xeloda® (capecitabine) in the xenograft model was assayed using 26 cell lines: DLD-1, LoVo, SW480, COLO201, WiDr, and CX-1 (all of the above are colon cancer cell lines); QG56, Calu-1, NCI-H441, and NCI-H596 (all of the above are lung cancer cell lines); MDA-MB-231, MAXF401, MCF7, ZR-75-1 (all of the above are breast cancer cell lines), AsPC-1, BxPC-3, PANC-1, and Capan-1 (all of the above are pancreatic cancer cell lines); MKN28 and GXF97 (all of the above are gastric cancer cell lines); SK-OV-3 and Nakajima (all of the above are ovarian cancer cell lines); Scaber and T-24 (bladder cancer cell line); Yumoto (uterine cancer cell line); and ME-180 (endometrial cancer cell line). The therapeutic experiment was carried out as follows. For example, in the case of LoVo (colon cancer cell line), 5.5×10⁶ cells were subcutaneously transplanted into nude mice. From the fifteenth day after the transplantation, the drug was orally administered at a dose of 2.1 mmole/kg/day to five mice from each group for five days a week; the oral administration was continued for four weeks. Based on the average tumor volume on the twenty-eighth day after the start of treatment (the day after the final administration), the tumor growth inhibition rate (TGI %) relative to the untreated group was determined as the in vivo sensitivity. For the remaining cell lines, the experiments were carried out according to the same method (FIG. 5).

Gene Expression Analysis

The experiment was carried out by the same procedure as in Example 1 using a DNA microarray.

Statistical Treatment

The respective values of tumor growth inhibition rate (TGI %) were converted to the categorized scores. Namely, score=2 for TGI %≧75; score=1 for 50≦TGI %<75; score=0 for TGI %<50.

The in vivo data obtained with the above-mentioned xenograft were used as the gene expression data. In the pre-treatment of gene expression data, as described above, the FC value was computed. Then, genes having standard deviations of FC equal to 2 or more and whose expression was found in 25% or more of the entire number of specimens used for the analysis were selected. By the pre-treatment, 2,929 genes were selected from the entire 12,559 genes. The correlation between expression data of 2,929 genes selected and the scored tumor growth inhibition rate was analyzed by the PLS1. The analysis resulted in a model consisting of five components, in which the square of the correlation coefficient (R²) was 1.00 and the square of the predictive correlation coefficient (Q²) was 0.47. The modeling power value (Ψ) was computed for every gene, and then genes with a value greater than 0.1 were selected as genes that highly contribute towards drug sensitivity. The PLS1 analysis was carried out again by using the expression data of the selected 821 genes and the tumor growth inhibition rate. The analysis resulted in a model consisting of five components, in which the square of the correlation coefficient (R²) was 1.00 and the square of the predictive correlation coefficient (Q²) was 0.77. The square of the predictive correlation coefficient (Q²) was drastically improved by the gene selection. Then, the genetic algorithm was used in order to thoroughly search for the combination of genes among the 821 genes where the Q² value is maximized and the number of genes selected is minimized. The evaluation function used is the following definition equation:
Evaluation function=Q²−α*K
where Q² represents the square of the predictive correlation coefficient in the PLS1 model; K represents the number of selected genes; α represents an appropriate penalty value.

According to a published report (Rogers et al. (1994) J. Chem. Inf. Comput. Sci. 34: 854-866), the genetic algorithm was conducted under the condition that the number of individuals is 400 and the number of generations is 100. The program based on the genetic algorithm was written in C language and was linked with PLS1 analysis software.

The analysis resulted in a model consisting of 82 genes and five components, in which the square of the correlation coefficient (R²) was 0.98 and the square of the predictive correlation coefficient (Q²) was 0.84. The value of standard deviations was 0.15. Thus, the reduction of the number of selected genes and the improvement of predictability (Q²) in the PLS model were successfully achieved by carrying out the model optimization in PLS1 analysis. This model consisting of 82 genes was taken as the final model.

Sensitivity Prediction

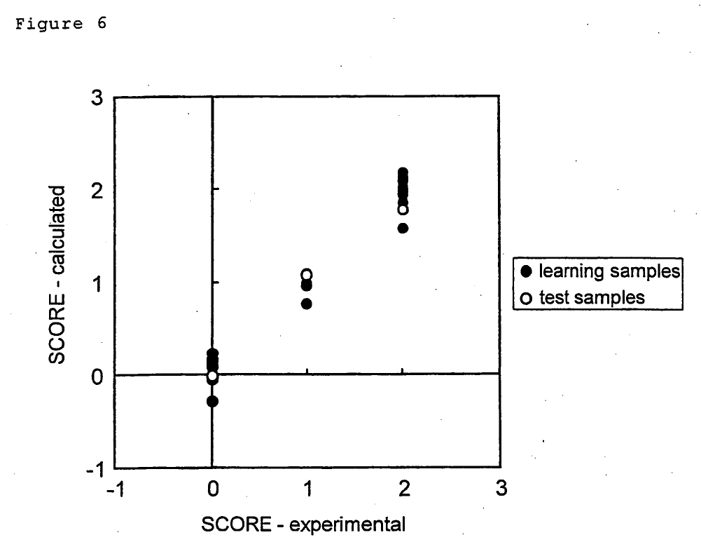

The score for each cell line, obtained by the computation based on the expression data of 82 genes identified, agreed well with the experimental value (FIG. 6). Based on the model, the antitumor effect was predicted in the three xenograft models for COLO205 (colon cancer cell line), MIAPaCa-2 (pancreatic cancer cell line), and MKN-45 (gastric cancer cell line); the predictability was very excellent, as seen in FIG. 6. The major group of selected genes and coefficient value in the PLS1model are shown in Tables 2 and 3, respectively. The Tables include the data of the thymidine phosphorylase gene as a positive contributing factor, known to correlate positively to the antitumor effect of Xeloda®, and thus the selection technique and model were demonstrated to be effective.

TABLE 2

| | | |
|---|---|---|
| GenBank | | |
| Ac. No. | Coefficient | Description |

Positive factors

| | | |
|---|---|---|
| Z35402 | 0.0257 | cadherin 1, type 1, E-cadherin (epithelial) |
| L19783 | 0.0215 | phosphatidylinositol glycan, class H |
| AF068706 | 0.0186 | adaptor-related protein complex 1, gamma 2 |
| | | subunit |
| AB007871 | 0.0182 | KIAA0411 gene product |
| AF033382 | 0.0167 | potassium voltage-gated channel, subfamily F, |
| | | member 1 |
| AF038198 | 0.0161 | chordin (CHRD) |
| AB007933 | 0.0154 | ligand of neuronal nitric oxide synthase with |
| | | carboxyl-terminal PDZ domain |
| M63193 | 0.013 | thymidine phosphorylase |
| AC004381 | 0.0126 | SA (rat hypertension-associated) homolog |
| U22376 | 0.0117 | v-myb avian myeloblastosis viral oncogene |
| | | homolog |
| M76676 | 0.0112 | leukocyte platelet-activating factor receptor |
| | | mRNA, complete cds |
| Z93096 | 0.0109 | manic fringe (Drosophila) homolog |
| AF054998 | 0.0107 | unknown function |

TABLE 3

| | | |
|---|---|---|
| GenBank | Co- | |
| Ac. No. | efficient | Description |

Negative factors

| | | |
|---|---|---|
| D90278 | −0.0375 | carcinoembryonic antigen-related cell adhesion |
| | | molecule 3 |
| AJ237672 | −0.026 | 5,10-methylenetetrahydrofolate reductase (NADPH) |
| AJ010063 | −0.0256 | titin-cap (telethonin) |
| M20777 | −0.0227 | alpha-2 (VI) collagen |
| M65066 | −0.0185 | protein kinase, cAMP-dependent, regulatory, type I, |
| | | beta |
| T92248 | −0.0174 | uteroglobin |
| M95925 | −0.0167 | neural retina leucine zipper |
| Y14153 | −0.0152 | beta-transducin repeat containing |
| AF014118 | −0.0144 | membrane-associated tyrosine-and threonine- |
| | | specific cdc2-inhibitory kinase |
| M60052 | −0.0141 | histidine-rich calcium-binding protein |
| J05213 | −0.0131 | integrin-binding sialoprotein (bone sialoprotein, |
| | | bone sialoprotein II) |
| X95694 | −0.0123 | transcription factor AP-2 beta (activating enhancer- |
| | | binding protein 2 beta) |
| D50683 | −0.0116 | striatin, calmodulin-binding protein |
| L36463 | −0.0102 | ras inhibitor |
| X74837 | −0.0101 | mannosidase, alpha, class 1A, member 1 |

### Industrial Applicability

According to the present invention, the therapeutic effect of an antitumor drug can be predicted for each patient prior to administration by a thorough analysis of gene expression in a small amount of specimens with unknown sensitivity, including cancer tissues. Thus, the present invention enables the selection of the most suitable drug for each patient (so-called tailor-made health care) and is useful for improving the patient's QOL.

1. A method for constructing a model that predicts sensitivity to a drug based on expression levels of genes, said method comprising the steps of:
(a) obtaining sensitivity data for a biological specimen;
(b) obtaining gene expression data for the biological specimen; and
(c) constructing a model by a partial least squares method type 1 using said sensitivity data obtained in step (a) and at least a part of said gene expression data for the biological specimen obtained in step (b), wherein said model can predict the sensitivity of the biological specimen to a specific drug.

2. The method according to claim 1, wherein, in the step (c), the model is optimized by constructing a model for each of two or more sets of combinations of genes by the partial least squares method type 1 and by selecting those models in which the number of genes is small and/or those models whose Q² value is high.

3. The method according to claim 2, wherein, in the step (c), the model is constructed by computing a parameter that represents a degree of contribution for each of the genes and by selecting the genes that have the greater relative parameter.

4. The method according to claim 3, wherein the parameter representing the degree of contribution is a modeling power value (Ψ).

5. The method according to claim 2, wherein, in the step (c), the model is constructed by generating different combinations of genes based on a genetic algorithm.

6. The method according to claim 1, wherein the sensitivity data comprises in vitro sensitivity data for a biological specimen.

7. The method according to claim 1, wherein the sensitivity data comprises animal-experimental sensitivity data for a biological specimen.

8. The method according to claim 1, wherein the sensitivity data comprises clinical sensitivity data for a biological specimen.

9. The method according to claim 1, wherein the drug is selected from the group consisting of the following farnesyltransferase inhibitors:
a) 6-[Amino-(4-chloro-phenyl)-(3-methyl-3H-imidazol-4-yl)-methyl]-4-(3-chloro-phenyl)-1-methyl-1H-quinolin-2-one; hydrochloride (Code: R115777);
b) (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile (Code: BMS214662);
c) (+)—(R)-4-[2-[4-(3,10-Dibromo-8-chloro-5,6-dihydro-11H-benzo[5,6]cyclohepta[1,2-b]pyridin-11-yl)piperidin-1-yl]-2-oxoethyl]piperidine-1-carboxamide (Code: SCH66336);
d) 4-[5-[4-(3-Chlorophenyl)-3-oxopiperazin-1-ylmethyl]imidazol-1-ylmethyl]benzonitrile (Code: L778123); and
e) 4-[hydroxy-(3-methyl-3H-imidazole-4-yl)-(5-nitro-7-phenyl-benzofuran-2-yl)-methyl]benzonitrile hydrochloride.

10. The method according to claim 1, wherein the drug is selected from the group consisting of the following fluorinated pyrimidines:
a) [1-(3,4-Dihydroxy-5-methyl-tetrahydro-furan-2-yl)-5-fluoro-2-oxo-1,2-dihydro-pyrimidin-4-yl]-carbamic acid butyl ester (Code: capecitabine (Xeloda®);
b) 1-(3,4-Dihydroxy-5-methyl-tetrahydro-furan-2-yl)-5-fluoro-1H-pyrimidine-2,4-dione (Code: Furtulon);
c) 5-Fluoro-1H-pyrimidine-2,4-dione (Code: 5-FU);
d) 5-Fluoro-1-(tetrahydro-2-furanyl)-2,4(1H,3H)-pyrimidinedione (Code: Tegafur);
e) a combination of Tegafur and 2,4(1H,3H)-pyrimidinedione (Code: UFT);
f) a combination of Tegafur, 5-chloro-2,4-dihydroxypyridine and potassium oxonate (molar ratio of 1:0.4:1) (Code: S-1); and
g) 5-Fluoro-N-hexyl-3,4-dihydro-2,4-dioxo-1 (2H)-pyrimidinecarboxamide (Code: Carmofur).

11. The method according to claim 1, wherein the drug is selected from the group consisting of the following taxanes:
a) [2aR-[2aα,4β,4aβ,6β,9α(αR*,βS*), 11α, 12β,12aα, 12bα]]-β-(benzoylamino)-α-hydroxybenzenepropanoic acid 6,12b-bis(acetyloxy)-12-(benzoyloxy)-2a,3,4,4a,5,6,9,10,11,12,12a, 12b-dodecahydro-4,11-dihydroxy-4a,8,13,13-tetramethyl-5-oxo-7,11-methano-1H-cyclodeca[3,4]benz[1,2-b]oxet-9-yl ester (Code: Taxol);
b) [2aR-[2aα,4β,4aα,6β,9α(αR*,βS*, 11α,12α,12aα,12bα)]-β-[[(1,1-dimethylethoxy)carbonyl]amino]-α-hydroxybenzenepropanoic acid 12b-(acetyloxy)-12-(benzoyloxy)-2a,3,4,4a,5,6,9,10,11,12,12a, 12b-dodecahydro-4,6,11-trihydroxy-4a,8,13,13-tetramethyl-5-oxo-7,11-methano-1H-cyclodeca[3,4]benz[1,2-b]oxet-9-yl ester (Code: Taxotere);
c) (2R,3 S)-3-[[(1,1-dimethylethoxy)carbonyl]amino]-2-hydroxy-5-methyl-4-hexenoic acid (3aS,4R,7R,8aS,9S, 10aR,12aS,12bR, 13S,13aS)-7,12a-bis(acetyloxy)-13-(benzyloxy)-3a,4,7,8,8a,9,10,10a, 12,12a, 12b, 13-dodecahydro-9-hydroxy-5,8a, 14,14-tetramethyl-2,8-dioxo-6,13a-methano-13aH-oxeto[2″,3″:5′,6′]benzo[1′,2′:4,5]cyclodeca[1,2-d]-11,3-dioxol-4-yl ester (Code: IDN 5109);
d) (2R,3 S)-β-(benzoylamino)-α-hydroxybenzenepropanoic acid (2aR,4S,4aS,6R,9S, 11 S,12S,12aR, 12bS)-6-(acetyloxy)-12-(benzoyloxy)-2a,3,4,4a,5,6,9,10,11,12,12a, 12b-dodecahydro-4,11-dihydroxy-12b-[(methoxycarbonyl)oxy]-4a,8,13,13-tetramethyl-5-oxo-7,11-methano-1H-cyclodeca[3,4]benz[1,2-b]oxet-9-yl ester (Code: BMS 188797); and
e) (2R,3 S)-β-(benzoylamino)-α-hydroxybenzenepropanoic acid (2aR,4S,4aS,6R,9S,11S,12S,12aR, 12bS)-6,12b-bis(acetyloxy)-12-(benzoyloxy)-2a,3,4,4a,5,6,9,10,11,12,12a,12b-dodecahydro-11-hydroxy-4a,8,13,13-tetramethyl-4-[(methylthio)methoxy]-5-oxo-7,11-methano-1H-cyclodeca[3,4]benz[1,2-b]oxet-9-yl ester (Code: BMS 184476).

12. The method according to claim 1, wherein the drug is selected from the group consisting of the following camptothecins:
a) 4(S)-ethyl-4-hydroxy-1H-pyrano[3′,4′:6,7]indolizino[1,2-b]quinoline-3,14(4H,12H)-dione (abbreviation: camptothecin);
b) [1,4′-bipiperidine]-1′-carboxylic acid, (4S)-4,11-diethyl-3,4,12,14-tetrahydro-4-hydroxy-3,14-dioxo-1H-pyrano[3′,4′:6,7]indolizino[1,2-b]quinolin-9-yl ester, monohydrochloride (Code: CPT-11);
c) (4S)-10-[(dimethylamino)methyl]-4-ethyl-4,9-dihydroxy-1H-pyrano [3′,4′:6,7]indolizino[1,2-b]quinoline-3,14(4H,12H)-dione monohydrochloride (abbreviation: Topotecan);
d) (1S,9S)-1-amino-9-ethyl-5-fluoro-9-hydroxy-4-methyl-2,3,9,10,13,15-hexahydro-1H,12H-benzo[de]pyrano[3′,4′:6,7]indolizino[1,2-b]quinoline-10,13-dione (Code: DX-8951f);
e) 5(R)-ethyl-9,10-difluoro-1,4,5,13-tetrahydro-5-hydroxy-3H,15H-oxepino[
3′,4′-6,7]indolizino[1,2-b]quinoline-3,15-dione (Code: BN-80915);
f) (S)-10-amino-4-ethyl-4-hydroxy-1H-pyrano[3′,4′:6,7]indolizino[1,2-b]quinoline-3,14(4H,12H)-dione (Code: 9-aminocamptotecin); and
g) 4(S)-ethyl-4-hydroxy-10-nitro-1H-pyrano[3′,4′,:6,7]-indolizino[1,2-b]quinol ine-3,14(4H, 12H)-dione (Code: 9-nitrocamptothecin).

13. The method according to claim 1, wherein the drug is selected from the group consisting of the following nucleoside analogue antitumor drugs:
a) 2′-deoxy-2′,2′-difluorocytidine (Code: DFDC);
b) 2′-deoxy-2′-methylidenecytidine (Code: DMDC);
c) (E)-2′-deoxy-2′-(fluoromethylene)cytidine (Code: FMDC);
d) 1-(β-D-arabinofuranosyl)cytosine (Code: Ara-C);
e) 4-amino-1-(2-deoxy-β-D-erythro-pentofuranosyl)-1,3,5-triazin-2(1H)-one (abbreviation: decitabine);
f) 4-amino-1-[(2S,4S)-2-(hydroxymethyl)-1,3-dioxolan-4-yl]-2(1H)-pyrimidinone (abbreviation: troxacitabine);
g) 2-fluoro-9-(5-O-phosphono-β-D-arabinofuranosyl)-9H-purin-6-amine (abbreviation: fludarabine); and
h) 2-chloro-2′-deoxyadenosine (abbreviation: cladribine).

14. The method according to claim 1, wherein the drug is selected from the group consisting of the following dolastatins:
a) N,N-dimethyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-L-valinamide (abbreviation: dolastatin 10);
b) cyclo[N-methylalanyl-(2E,4E, 10E)-15-hydroxy-7-methoxy-2-methyl-2,4,10-hexadecatrienoyl-L-valyl-N-methyl-L-phenylalanyl-N-methyl-L-valyl-N-methyl-L-valyl-L-prolyl-N-2-methylasparaginyl] (abbreviation: dolastatin 14);
c) (1 S)-1-[[(2S)-2,5-dihydro-3-methoxy-5-oxo-2-(phenylmethyl)-1H-pyrrol-1-yl]carbonyl]-2-methylpropyl ester N,N-dimethyl-L-valyl-L-valyl-N-methyl-L-valyl-L-prolyl-L-proline (abbreviation: dolastatin 15);
d) N,N-dimethyl-L-valyl-N-[(1S,2R)-2-methoxy-4-[(2S)-2-[(1R,2R)-1-methoxy-2-methyl-3-oxo-3-[(2-phenylethyl)amino]propyl]-1-pyrrolidinyl]-1-[(1S)-1-methylpropyl]-4-oxobutyl]-N-methyl-L-valinamide (Code: TZT 1027); and
e) N,N-dimethyl-L-valyl-L-valyl-N-methyl-L-valyl-L-prolyl-N-(phenylmethyl)-L-prolinamide (abbreviation: cemadotin).

15. The method according to claim 1, wherein the drug is selected from the group consisting of the following anthracyclinesanthracyclines:
a) (8S,10S)-10-[(3-amino-2,3,6-trideoxy-L-lyxo-hexopyranosyl)oxy]-7,8,9,10-tetrahydro-6,8,11-trihydroxy-8-(hydroxyacetyl)-1-methoxynaphthacene-5,12-dione hydrochloride (abbreviation: adriamycin);
b) (8S, 10S)-10-[(3-amino-2,3,6-trideoxy-L-arabino-hexopyranosyl)oxy]-7,8,9,10-tetrahydro-6,8,11-trihydroxy-8-(hydroxyacetyl)-1-methoxynaphthacene-5,12-dione hydrochloride (abbreviation: epirubicin);
c) 8-acetyl-10-[(3-amino-2,3,6-trideoxy-L-lyxo-hexopyranosyl)oxy]-7,8,9,10-tetrahydro-6,8,11-trihydroxy-1-methoxynaphthacene-5,12-dione, hydrochloride (abbreviation: daunomycin); and
d) (7S,9S)-9-acetyl-7-[(3-amino-2,3,6-trideoxy-L-lyxo-hexopyranosyl)oxy]-7,8,9,10-tetrahydro-6,9,11-trihydroxynaphthacene-5,12-dione (abbreviation: idarubicin).

16. The method according to claim 1, wherein the drug is selected from the group consisting of the following protein kinase inhibitors:
a) N-(3-chloro-4-fluorophenyl)-7-methoxy-6-[3-(4-morpholinyl)propoxy]-4-quinazolinamine (Code: ZD 1839);
b) N-(3-ethynylphenyl)-6,7-bis(2-methoxyethoxy)-4-quinazolinamine (Code: CP 358774);
c) N⁴-(3-bromophenyl)-N-6-methylpyrido[3,4-d]pyrimidine-4,6-diamine (Code: PD 158780);
d) N-(3-chloro-4-((3-fluorobenzyl)oxy)phenyl)-6-(5-(((2-methylsulfonyl)ethyl)amino)methyl)-2-furyl)-4-quinazolinamine (Code: GW 2016);
e) 3-[(3,5-dimethyl-1H-pyrrol-2-yl)methylene]-1,3-dihydro-2H-indol-2-one (Code: SU5416);
f) (Z)-3-[2,4-dimethyl-5-(2-oxo-1,2-dihydro-indol-3-ylidenemethyl)-1H-pyrrol-3-yl]-propionic acid (Code: SU6668);
g) N-(4-chlorophenyl)-4-(pyridin-4-ylmethyl)phthalazin-1-amine (Code: PTK787);
h) (4-bromo-2-fluorophenyl) [6-methoxy-7-(1-methylpiperidin-4-ylmethoxy)quinazolin-4-yl]amine (Code: ZD6474);
i) N⁴-(3-methyl-1H-indazol-6-yl)-N-(3,4,5-trimethoxyphenyl)pyrimidine-2,4-diamine (Code: GW2286);
j) 4-[(4-methyl-1-piperazinyl)methyl]-N-[4-methyl-3-[[4-(3-pyridinyl)-2-pyrimidinyl]amino]phenyl]benzamide (Code: STI-571);
k) (9α, 10β,11β,13α)-N-(2,3,10,12,13-hexahydro-10-methoxy-9-methyl-1-oxo-9,13-epoxy-1H,9H-diindolo[1,2,3-gh:3′,2′,1′-1m]pyrrolo[3,4-j][1,7]benzodiazonin-11-yl)-N-methylbenzamide (Code: CGP41251);
l) 2-[(2-chloro-4-iodophenyl)amino]-N-(cyclopropylmethoxy)-3,4-difluorobenzamide (Code: C11040); and
m) N-(4-chloro-3-(trifluoromethyl)phenyl)-N′-(4-(2-(N-methylcarbamoyl)-4-pyridyloxy)phenyl)urea (Code: BAY439006).

17. The method according to claim 1, wherein the drug is selected from the group consisting of the following platinum antitumor drugs:
a) cis-diaminodichloroplatinum(II) (abbreviation: cisplatin);
b) diammine(1,1-cyclobutanedicarboxylato)platinum(II) (abbreviation: carboplatin);
and
c) hexaamminedichlorobis[μ-(1,6-hexanediamine-κN: κN′)]tri-,stereoisomer,tetranitrate platinum(4+) (Code: BBR3464).

18. The method according to claim 1, wherein the drug is selected from the group consisting of the following epothilones:
a) 4,8-dihydroxy-5,5,7,9,13-pentamethyl-16-[(1E)-1-methyl-2-(2-methyl-4-thiazolyl)ethenyl]-(4S,7R,8S,9S,13Z,16S)-oxacyclohexadec⁻¹³-ene-2,6-dione (abbreviation: epothilone D);
b) 7,11-dihydroxy-8,8,10,12,16-pentamethyl-3-[(1E)-1-methyl-2-(2-methyl-4-thiazolyl)ethenyl]-, (1S,3S,7S,10R,11S,12S,16R)-4,17-dioxabicyclo[14.1.0]heptadecane-5,9-dione6-dione (abbreviation: epothilone); and
c) (1S,3S,7S,10R,11S,12S,16R)-7,11-dihydroxy-8,8,10,12,16-pentamethyl-3-[(1E)-1-methyl-2-(2-methyl-4-thiazolyl)ethenyl]-17-oxa-4-azabicyclo[14.1.0]heptadecane-5,9-dione (Code: BMS247550).

19. The method according to claim 1, wherein the drug is selected from the group consisting of the following aromatase inhibitors:
a) α,α,α′,α′-tetramethyl-5-(1H-1,2,4-triazol-1-ylmethyl)-1,3-benzenediacetonitrile (Code: ZD1033);
b) (6-methyleneandrosta-1,4-diene-3,17-dione (Code: FCE24304); and
c) 4,4′-(1H-1,2,4-triazol-1-ylmethylene)bis-benzonitrile (Code: CGS20267).

20. The method according to claim 1, wherein the drug is selected from the group consisting of the following hormone modulators:
a) 2-[4-[(1 Z)-1,2-diphenyl-1-butenyl]phenoxy]-N,N-dimethylethanamine (abbreviation: tamoxifen);
b) [6-hydroxy-2-(4-hydroxyphenyl)benzo[b]thien-3-yl][4-[2-(1-piperidinyl)ethoxy]phenyl]methanone hydrochloride (Code: LY156758);
c) 2-(4-methoxyphenyl)-3-[4-[2-(1-piperidinyl)ethoxy]phenoxy]benzo[b]thiophene-6-ol hydrochloride (Code: LY3553381);
d) (+)-7-pivaloyloxy-3-(4′-pivaloyloxyphenyl)-4-methyl-2-(4″-(2″′-piperidinoethoxy)phenyl)-2H-benzopyran (Code: EM800);
e) (E)-4-[1-[4-[2-(dimethylamino)ethoxy]phenyl]-2-[4-(1-methylethyl)phenyl]-1-butenyl]phenol dihydrogen phosphate(ester) (Code: TAT59);
f) 17-(acetyloxy)-6-chloro-2-oxapregna-4,6-diene-3,20-dione (Code: TZP4238);
g) (+,−)-N-[4-cyano-3-(trifluoromethyl)phenyl]-3-[(4-fluorophenyl)sulfonyl]-2-hydroxy-2-methylpropanamide (Code: ZD 176334); and
h) 6-D-leucine-9-(N-ethyl-L-prolinamide)-10-deglycinamide luteinizing hormone-releasing factor (pig) (abbreviation: leuprorelin).

21. The method according to claim 1, wherein the biological specimen is a cancer cell or a cancer cell line.

22. The method according to claim 1, wherein the sensitivity comprises an antitumor effect.

23. The method according to claim 1, wherein the gene expression data comprises high-density nucleic acid array data.

24. A method for selecting genes that contribute to biological sensitivity to a high degree, said method comprising the step of selecting part or all of the combinations of genes in a model constructed by the method according claim 1.

25. A method for predicting the sensitivity of a test specimen toward a particular stimulus, said method comprising the steps of:
(a) obtaining, for the test specimen, at least a part of a gene expression data from a modelspecimen constructed by the method according to claim 1; and
(b) correlating to the fact that the sensitivity is high, a high level of expression of a gene having a positive coefficient in the model and a low level of expression of a gene having a negative coefficient in the model, and correlating to the fact that the sensitivity is low, a low level of expression of a gene having a positive coefficient in the model and a high level of expression of a gene having a negative coefficient in the model.

26. The method according to claim 25, wherein:
step (a) comprises the step of obtaining the gene expression data in the model for the test specimen; and
step (b) comprises the step of computing the sensitivity by applying the expression data to the model.

27. A computer device that predicts the sensitivity of a test specimen toward a particular stimulus, said device comprising:
(a) a means for storing a parameter (model coefficient) representing the relationship between gene expression data and sensitivity value in a model constructed by the method according to claim 1;
(b) a means for inputting the gene expression data into the model;
(c) a means for storing the expression data;
(d) a means for predictively calculating the sensitivity value from the expression data and the parameter (model coefficient) based on the model;
(e) a means for storing the predictively calculated sensitivity value; and
(f) a means for outputting the predictively calculated sensitivity value or a result obtained from the sensitivity value.

28. A method for producing a high-density nucleic acid array, said method comprising the step of immobilizing or generating, on a support, nucleic acids comprising at least 15 nucleotides comprised in nucleotide sequences encoding respective genes selected by the method according to claim 24.

29. A method for producing a probe or a primer for quantitative or semi-quantitative PCR for respective genes selected by the method according to claim 24, said method comprising the step of synthesizing nucleic acids comprising at least 15 nucleotides comprised in nucleotide sequences encoding the respective genes.

30. A kit comprising:
(a) a high-density nucleic acid array, or a probe or a primer for quantitative or semi-quantitative PCR, wherein said array, probe, or primer comprises nucleic acids comprising at least 15 nucleotides from nucleotide sequences encoding respective genes selected by the method according to claim 24; and
(b) a storage medium which records the sensitivity to drugs predicted using the array, or the probe or the primer.

31. A method for selecting genes that contribute to biological sensitivity to a high degree, said method comprising the step of selecting part or all of the combinations of genes in a model constructed by the method according to claim 2.
